(19) United States
(12) Patent Application Publication
(10) Pub. No.: US 2026/0275337 A1
(43) Pub. Date: Sep. 17, 2026

(54) ANTISENSE OLIGONUCLEOTIDES FOR PREVENTING KCNQ2 MISPLICING

(71) Applicants: Northwestern University, Evanston, IL (US); UNIVERSITY OF MASSACHUSETTS, Westborough, MA (US)

(72) Inventors: Evangelos Kiskinis, Evanston, IL (US); Wanhao Chi, Evanston, IL (US); Jonathan Watts, Worcester, MA (US)

(73) Assignees: Northwestern University, Evanston, IL (US); UNIVERSITY OF MASSACHUSETTS, Westborough, MA (US)

(21) Appl. No.: 19/168,295

(22) PCT Filed: Mar. 24, 2024
(86) PCT No.: PCT/US2024/021258

§ 371 Date: Sep. 23, 2025

Related U.S. Application Data
(60) Provisional application No. 63/454,540, filed on Mar. 24, 2023.

(51) Int. Cl. C12N 15/113; A61P 25/28

(52) U.S. Cl. CPC C12N 15/113; A61P 25/28; C12N 2310/11; C12N 2310/315; C12N 2310/321

(57) ABSTRACT

Provided herein are antisense oligonucleotides comprising, consisting of, or consisting essentially of 10 to 30 nucleotides, wherein the ASO binds to a target region comprising an intronic sequence between exons 4 and 6 of a nucleic acid molecule encoding a human KCNQ2 protein.

### CROSS REFERENCE TO RELATED APPLICATIONS

This application is the U.S. National Stage of PCT/US2024/021258, filed Mar. 24, 2024, which claims the benefit under 35 U.S.C. § 119(e) of U.S. Provisional Application Ser. No. 63/454,540, filed Mar. 24, 2023, the contents of which are hereby incorporated by reference in its entirety.

The instant application contains a Sequence Listing which has been submitted in XML format and is hereby incorporated by reference in its entirety. Said XML copy, created on May 21, 2024, is named 121384-0227.xml and is 54,020 bytes.

### TECHNICAL FIELD

The present technology relates generally to treatments with antisense oligonucleotides. More specifically, the present technology relates to the treatment of neurodegenerative disorders with antisense oligonucleotides.

### BACKGROUND

The following description of the background of the present technology is provided simply as an aid in understanding the present technology and is not admitted to describe or constitute prior art to the present technology.

Amyotrophic lateral sclerosis (ALS) is a fatal neurodegenerative disorder perturbing upper and lower motor neurons (MNs) that are responsible for skeletal muscle contraction and locomotion. While the disease is inherited in 10% of cases, the broad heterogeneity of ALS is underscored by the majority of patients in whom it arises sporadically. Consequently, there remain significant challenges in identifying disease-specific biomarkers for early diagnosis and therapeutic targets. The cytoplasmic mislocalization and aggregation of TDP-43 (TAR DNA-binding protein of 43 kDa) represents a shared histopathological feature that is found in over 90% of all ALS cases. TDP-43 trans-localization and cytoplasmic aggregation also occurs in approximately 50% of frontotemporal dementia (FTD-TDP) patients. TDP-43 is a nuclear RNA binding protein (RBP) with conserved roles across the RNA lifecycle, from transcriptional regulation to RNA splicing modulation, RNA transport and stability. The link between pathology and neurodegeneration was codified by the identification of rare mutations in TARDBP, the gene that encodes TDP-43 in ALS families. However, the cellular mechanisms that relate TDP-43 with neuronal dysfunction and neurotoxicity remain unclear.

Neuronal hyperexcitability is a broadly observed clinical feature in familial and sporadic amyotrophic lateral sclerosis (ALS) patients, yet the underlying causes remain unknown. Nuclear depletion and cytoplasmic aggregation of the RNA splicing modulator TDP-43 is a unifying neuropathological feature of most ALS patients.

The recent interrogation of human induced pluripotent stem cell (iPSC)-derived neurons with loss of TDP-43 function and postmortem ALS patient tissue has revealed several dozen target mRNAs that become mis-spliced. The pre-mRNA encoding Stathmin2 (STMN2), a modulator of microtubule stability that impacts axonal outgrowth, becomes mis-processed and yields a cryptic last exon that results in a premature stop codon and transcript degradation. Similarly, TDP-43 depletion causes the inclusion of a cryptic exon in KCNQ2, which encodes a protein with critical functions in synaptic vesicle release, resulting in nonsense mediated decay and downregulation of the protein. Intriguingly, polymorphisms in KCNQ2 that overlap with TDP-43 binding sites, are strongly associated with increased ALS and FTD risk. These findings have shifted the focus from gain-of-function effects associated with aggregated TDP-43 to loss-of-function effects driven by its nuclear depletion, as they underscore direct relationships between TDP-43 pathology, neuronal function, and disease risk. However, a link between the role of TDP-43 in splicing and commonly observed clinical features of ALS/FTD patients has not yet been established.

Neuronal hyperexcitability is a broadly observed clinical feature in familial and sporadic ALS. Motor neuron excitability, measured by transcranial magnetic stimulation (TMS) and threshold tracking nerve conduction studies (TTNCSs), is typically an early indicator of MN disease, and greater excitability is prognostic of disease progression and survival. Excitability disturbances have been linked to a loss of cortical inhibition and changes in the expression and activity of key ion channels that serve to regulate intrinsic membrane potential. In support of the latter, hyperexcitability has been observed in iPSC-based ALS models and can be modulated by retigabine (also known as ezogabine), a relatively selective activator of Kv7 channels, which are known to be widely expressed in the nervous system and play a central role in regulating excitability. Notably, treatment of ALS iPSC-MNs with ezogabine reduced endoplasmic reticulum (ER) stress and improved survival suggesting a mechanistic interaction between excitability and proteotoxic degeneration. The anti-epileptic drug ezogabine has also been shown to effectively decrease both cortical and spinal MN excitability in ALS patients in a phase 2 clinical trial, which was not designed to assess efficacy. Despite its wide prevalence in the patient population and therapeutic potential, the mechanisms that drive intrinsic neuronal hyperexcitability in ALS are poorly understood.

### SUMMARY OF THE PRESENT TECHNOLOGY

The present disclosure provides ASOs that prevent mis-splicing of KCNQ2 and treatments utilizing the same.

In one aspect, the present disclosure provides an antisense oligonucleotide (ASO), comprising, consisting of, or consisting essentially of 10 to 30 nucleotides. The ASO binds to a target region comprising, consisting of, or consisting essentially of an intronic sequence between exons 4 and 6 of a nucleic acid molecule encoding a human KCNQ2 protein. In some aspects, the ASO is modified or unmodified

In one aspects, the target region comprises, consists of, or consists essentially of a nucleic acid sequence of:

(SEQ ID NO: 19)
TGGTCATTGTGGTTTTCCCTTTCCCTGCTGATACACCCCTGTCCCTGTG
CTGGGACCAGGCTCTCACTGGCTGAGCCTGCTCCATACATCTCTTTGGG
GCCACCTGCTGGCTGCCCGTGGTCATGATGGCTTGTGGTCGAGGCGGGG
TGGTGGTTGCCAAGCTGGCGGGGGAGTGGGTGGGGAGGGCTGGGCCCCA
GAGAGCCTGAGCTCAGCCGTGGTGGGCATCTTCCTTCCTCCTGAGCTCA
GCCGTGGTGGGCATCTTCCTTCCTCCTGAGTGACCCTCCACCTGCTCCG
TGTCCCGATTATTGGGG
or
(SEQ ID NO: 20)
TGGTGGTGGTGGTGATGGTCATGATGGTCATGGTGGTGGTGGTGGTGAT
GGTGATGGTGGTGATCGTGGTGGTGGTGATGGTGATGGTGGTGGTGATG
ATGGTGGTGATGGTGGTGATGGTGATGGTGGTGGTGATGGTGATGGTGA
T.

In some aspects, ASO comprises, consists of, or consists essentially of a nucleotide sequence selected from: CACCATCACCATCACCACCA (SEQ ID NO: 21) or a pharmaceutically acceptable salt thereof; ACCATCACCATCACCACCA (SEQ ID NO: 22) or a pharmaceutically acceptable salt thereof; ATCACCATCACCACCACCA (SEQ ID NO: 23) or a pharmaceutically acceptable salt thereof; ACCATCACCACCACCACCA (SEQ ID NO: 24) or a pharmaceutically acceptable salt thereof; ATCACCATCACCATCACCA (SEQ ID NO: 25) or a pharmaceutically acceptable salt thereof; CACCACCATCATCACCAC (SEQ ID NO: 33) or a pharmaceutically acceptable salt thereof; CACCATCACCATCACCA (SEQ ID NO: 26) or a pharmaceutically acceptable salt thereof; ATCACCATCACCATCA (SEQ ID NO: 27) or a pharmaceutically acceptable salt thereof; AGGGAAAACCACAATGACCA (SEQ ID NO: 28) or a pharmaceutically acceptable salt thereof; CCATCATGACCACGGGCAGCC (SEQ ID NO: 29) or a pharmaceutically acceptable salt thereof; ATGACCACGGGCAGCCAGCA (SEQ ID NO: 30) or a pharmaceutically acceptable salt thereof; ACCACGGCTGAGCTCAGGCTCT (SEQ ID NO: 31) or a pharmaceutically acceptable salt thereof, or TCACTCAGGAGGAAGGAAGAT (SEQ ID NO: 32) or a pharmaceutically acceptable salt thereof.

In some aspects, the ASO comprises, consists of, or consists essentially of at least one modified internucleoside linkage. In some aspects, the internucleoside linkage comprises, consists of or consists essentially of a wherein the modified internucleoside linkage is a phosphorothioate linkage. In some aspects, each of the internucleoside linkages in the ASO comprise, consist of, or consist essentially of a phosphorothioate linkage. In yet another aspect, at least one of the nucleotides in the ASO comprises, consist of, or consists essentially of a modified nucleotide. In some aspects, all of the nucleotides in the ASO comprise, consist of, or consist essentially of modified nucleotides, the modified nucleotide(s) comprise, consist of, or consist essentially of 2′-O-methoxyethyl (MOE) nucleotide(s).

In some aspects, the nucleic acid molecule encoding a human KCNQ2 protein comprises, consists of or consists essentially of an mRNA.

In some aspects, provided herein is a pharmaceutical composition comprising, consisting of, or consisting essentially of the ASO and a carrier.

In yet another aspect, provided herein is method of preventing KCNQ2 mis-splicing in a cell, comprising, consisting of, or consisting essentially of contacting the cell with the ASO or the pharmaceutical composition.

In yet another aspect, provided herein is method of treating or preventing a neurodegenerative disease in a subject, comprising, consisting of, or consisting essentially of contacting the cell with the ASO or the pharmaceutical composition. In some aspects, the neurodegenerative disease involves TDP-43 cytoplasmic aggregation, TDP-43 nuclear depletion, or both. In some aspects, the neurodegenerative disease comprises, consists of, or consists essentially of Amyotrophic Lateral Sclerosis (ALS) or frontotemporal dementia (FTD). In some aspects, the subject is a human.

### BRIEF DESCRIPTION OF THE DRAWINGS

FIGS. 1A-1I: TDP-43 depletion in neurons leads to skipping of a constitutively expressed exon in the epilepsy gene, KCNQ2. (FIG. 1A) Differential splicing analysis of stem-cell-derived, purified MNs treated with TDP-43-targeting (n=6) or scrambled (n=6) siRNAs previously reported in Klim et al9. Annotated (known) alternative splicing events are represented in green whereas de novo events that arise specifically because of loss of TDP-43 activity are highlighted in orange. A de novo KCNQ2 splicing event is highlighted in red. (FIG. 1B) Heatmap and classification of splicing events that are altered with TDP-43 depletion. Percent Spliced Index (PSI) quantifies proportion of reads mapping to one splice variant relative to all splice variants from the same locus. de novo junctions are splice variants that are not normally expressed at detectable levels. (FIG. 1C) Identification of pervasively mis-spliced genes involved in the regulation of membrane potential. Left: Integration of TDP-43 splicing targets derived from purified MN9, i3Neurons27, and TDP-43-low neuronal nuclei29 yields 522 genes, 47 of which are involved in the regulation of membrane potential and/or synaptic signaling. Right: Only 2 genes (KCNQ2 and CACNA1E) are consistently mis-spliced in all three datasets. (FIG. 1D) Sashimi plot depicting HISAT2-mapped sequencing coverage along exons 4 through 6 of KCNQ2, which has defective splicing under TDP-43 depletion. The gene models below indicate the locations of exons and introns. Junction spanning reads mapping to the canonical junctions (exons 4-5 and exons 5-6) are indicated with solid lines, whereas de novo junction (exons 4-6) reads are displayed with a dashed line. Read counts and PSI scores supporting each splice junction are denoted. Note that the de novo KCNQ2 exon skipping event is only supported under TDP-43 depletion. (FIG. 1E) RT-PCR assay reveals aberrant processing of KCNQ2 in cortical neurons depleted of TDP-43 by siRNA. (FIG. 1F) Quantitative RT-PCR (qRT-PCR) assay reveals the magnitude of aberrant processing of KCNQ2 in cortical neurons depleted of TDP-43 by siRNA. (FIG. 1G) qRT-PCR assay reveals that loss of TDP-43 does not alter total KCNQ2 abundance. (FIG. 1H-FIG. 1J) TDP-43 directly interacts with the KCNQ2 pre-Mrna. (FIG. 1H) iCLIP coverage27 in the KCNQ2 locus suggests TDP-43 binds KCNQ2. TDP-43 consensus motifs (UGNNUG) and (UG) n simple repeat are shown below the KCNQ gene model. qRT-PCR amplicon used in I-J is indicated. (FIG. 11) Crosslinking immunoprecipitation followed by RT-PCR analysis was used to assay enrichment of KCNQ2 in TDP-43-bound fraction compared to IGG immunoprecipitates using the same quantity of lysate for IPs. (J) Quantification of results present in (I).

FIGS. 2A-2M: Identification and prioritization of TDP-43 dependent KCNQ2 mis-splicing. (FIG. 2A) Overlap of 125 differential local splice variants (LSVs) detected in purified MNs with TDP-43 knockdown9, with other datasets of TDP-43 depletion reported in Liu et al29 and Brown et al27. (FIG. 2B) related to FIG. 1C. Left: of the 522 genes with altered splicing in neuronal datasets, 47 are involved in the regulation of membrane potential and synaptic signaling. Right: list of 47 genes. Those with de novo events, such as cryptic exon derepression are shown in red. (FIG. 2C) Sashimi plot depicting HISAT2-mapped sequencing coverage and junction spanning reads of alternatively spliced locus in CACNA1E. Inclusion of annotated cassette exon increases following TDP-43 loss. (FIG. 2D-FIG. 2E) Genes involved in the regulation of membrane potential and synaptic signaling are not commonly differentially expressed (DE) in neurons depleted of TDP-43. (FIG. 2D) Total number of DE genes in each set of neuronal datasets. (FIG. 2E) Number of DE genes in each set, involved in the regulation of membrane potential and synaptic signaling. (FIG. 2F) Overlap of DE genes identified in (FIG. 2E). (FIG. 2G) top: KCNQ2 gene model, including location of skipped exon 5 and RT-PCR primers. Bottom: exon 5 is deeply conserved. Graphic is generated using the UCSC genome browser, where each dot indicates conservation of the related amino acid. FIG. 2G discloses SEQ ID NOS: 1-11, respectively, in order of appearance. (FIG. 2H) Amino acid sequence alignment for wildtype and KCNQ2ΔE5. A fragment of the peptide is shown, and individual domains are boxed, shaded and annotated. Exon 5 codes for transmembrane domain 5, extracellular components and a portion of the intramembrane pore-forming domain. FIG. 2H discloses SEQ ID NOS: 12-15, respectively, in order of appearance. (FIG. 2I-FIG. 2J) Splicing of murine KCNQ2 is not regulated by TDP-43-Data from Polymenidou et al39. (FIG. 2I) Sashimi plot depicting HISAT2-mapped sequencing coverage and junction spanning reads of syntenic mouse KCNQ2 locus. There is no exon skipping following TDP-43 reduction (TDP-43 ASO). (FIG. 2J) TDP-43 depletion in mouse brains treated with TDP-43 targeting ASOs. (FIG. 2K) Verification of mis-spliced KCNQ2 in DIV 40 human iPSC-MNs. (FIG. 2L) Confirmation of mis-spliced KCNQ2 by Sanger sequencing. FIG. 2L discloses SEQ ID NOS: 16-18, respectively, in order of appearance. (FIG. 2M) Abundance of KCNQ2ΔE5 increases with stronger knockdown of TDP-43. Comparison of KCNQ2ΔE5 and TDP-43 abundance by reanalysis of a collection reported datasets9,27.

FIGS. 3A-3K: Expression of KCNQ2ΔE5 is highly specific to ALS/FTD and TDP-43 pathology. (FIG. 3A) RNA-seq datasets obtained from the Target ALS Human Postmortem Tissue Core at the NYGC consisted of 118 non-neurological controls, 965 ALS samples and 41 other neurological disease samples derived from distinct CNS regions. (FIG. 3B) Strategy to detect reads supporting the KCNQ2ΔE5 transcript. (FIG. 3C) Reads mapping to KCNQ2ΔE5 in RNA-seq datasets stratified by disease status reveals that KCNQ2ΔE5 is highly specific to ALS. Each point represents a unique sample, and the score indicates number of unique KCNQ2ΔE5 reads (FIG. 3D) Percentage of ALS samples from distinct CNS regions with detectable KCNQ2ΔE5. Intensity of red shade is relative to proportion of samples with detectable KCNQ2ΔE5. (FIG. 3E) Reads mapping to KCNQ2ΔE5 in RNA-seq datasets stratified by patient genetics reveals that KCNQ2ΔE5 is highly specific to TDP-43 pathologies. As in (FIG. 3C), each point represents a unique sample, and the score indicates number of unique KCNQ2ΔE5 reads. (FIG. 3F-FIG. 3I) Analysis of KCNQ2 in patient brain samples with TDP-43 pathology or controls with qRT-PCR using primers that specifically amplify KCNQ2ΔE5 (FIG. 3F) and wildtype KCNQ2 (FIG. 3G). (FIG. 3H) KCNQ2ΔE5 abundance increases with TDP-43 pathology, as measured by phosphorylated TDP-43 levels. (FIG. 3I) KCNQ2ΔE5 abundance is higher in individuals with late disease onset. (J-K) Abundance of KCNQ2ΔE5 in published datasets of TDP-43 high and low neuronal nuclei from FTLD/ALS-FTLD patients with C9orf72 hyperexpansions29. (FIG. 3J) top: schematic of neuronal nuclei from patient brains sorted by flow cytometry into fractions with high and low TDP-43 levels. Below: Sashimi plot depicting HISAT2-mapped sequencing coverage along exons 4 through 6 of KCNQ2. The gene models below indicate the locations of exons and introns. Junction spanning reads mapping to the canonical junctions (exons 4-5 and exons 5-6) are indicated with solid blue lines, whereas KCNQ2 exon 5 skipping reads are displayed with red dashed lines. Read counts supporting splice junctions are denoted. Note that the aberrant KCNQ2 exon skipping event is detected in datasets from all individuals, but only in TDP-43 low neurons. (FIG. 3K) PSI quantification of reads in (J) indicates that approximately 50% of KCNQ2 is mis-spliced in the nuclei of TDP-43 low neurons.

FIGS. 4A-4F: Expression of KCNQ2ΔE5 is highly specific to ALS/FTD and TDP-43 pathology. (FIG. 4A) Reads mapping to KCNQ2ΔE5 in RNA-seq datasets stratified by disease status and CNS tissue source. Each point represents a unique sample, and the score indicates number of unique KCNQ2ΔE5 reads. (FIG. 4B) Boxplots displaying abundance of select genes (KCNQ2, STMN2 and UNC13A) in different CNS regions. Each box reveals the interquartile range of gene expression. (FIG. 4C) Proportion of regional CNS tissue samples with aberrant splicing events. Left: KCNQ2ΔE5, Middle: cryptic truncation of STMN2 and Right: cryptic exon inclusion in UNC13A. Numbers of samples with and without detection of events are included (Detected: not detected). (FIG. 4D-FIG. 4E) Expression of KCNQ2, STMN2 and UNC13A in different cell types in the human cortex. Data from Schirmer et al43 (FIG. 4D) and Velmeshev et al44 (FIG. 4E). (FIG. 4F) Related to FIG. 2F-2I. Correlation between KCNQ2ΔE5 abundance by qRT-PCR and disease duration.

FIGS. 5A-5G: Expression of KCNQ2ΔE5 causes intracellular accumulation and non-functional Kv7 channels. (FIG. 5A) Amino acid sequence (220-280) and corresponding subunit illustration for wildtype KCNQ2 in comparison to TDP-43 associated KCNQ2ΔE5 and dominant negative epilepsy variant G279S. Deletion of exon 5 likely results in unique protein structure, represented here by a dashed line. Single nucleotide variant G279S resides within the pore loop, approximate location denoted as a star symbol. FIG. 5A discloses SEQ ID NOS: 47-49, respectively, in order of appearance. (FIG. 5B) Schematic illustration outlining heterologous expression system and subsequent Syncropatch experiments. Human KCNQ2 transgenes are transiently transfected into KCNQ3-expressing CHO-K1 cells. hKCNQ2 transgenes can be expressed alone to recapitulate a homozygous (left) or in equal amounts with KCNQ2WT to mimic a heterozygous state (right). Transfected cells are then plated into a 384-well plate where automated voltage-clamp experiments, represented inside dashed blue line, occur simultaneously in each well. One cell per well adheres to the plate and access to the internal solution and electrode is achieved. Current generated from cells expressing KCNQ2 channels is recorded across a range of command voltages. (FIG. 5C) Syncropatch results for homozygous expression of hKCNQ2 transgenes. (left) Summary data (mean±SEM) for outward current density as a function of command voltage (KCNQ2WT: n=85, KCNQ2ΔE5: n=51, KCNQ2G279S: n=53); (right) Representative KCNQ2 current (XE-991-sensitive) traces for voltage-clamp protocol. No observable current is observed from either KCNQ2ΔE5 or KCNQ2G279S when expressed alone with KCNQ3. (FIG. 5D) Syncropatch results for heterozygous expression of hKCNQ2 transgenes; (left) Summary data (mean±SEM) for outward current density as a function of command voltage (KCNQ2WT (×2): n=51, KCNQ2WT+KCNQ2ΔE5: n=59, KCNQ2WT+KCNQ2G279S: n=60); (right) Representative KCNQ2 current (XE-991-sensitive) traces for voltage-clamp protocol. Outward current can be recorded when KCNQ2 variants are co-expressed with KCNQ2WT. KCNQ2G279S is a known dominant-negative variant, observed here as a >50% reduction from KCNQ2WT current. KCNQ2ΔE5 produces a slight reduction. (FIG. 5E) Immunocytochemical labeling of CHO-K1 cells expressing hKCNQ2 GFP/RFP lentiviral transgenes. Top panel corresponds to cells expressing GFP-KCNQ2WT and RFP-KCNQ2WT. GFP-KCNQ2WT and GFP-KCNQ2ΔE5 signal metrics were extracted for each cell using ROI drawn from max intensity projections (GFP-WT: n=19, DelE5: n=19); scale bar=2 m. (FIG. 5F) Quantification of maximal GFP signal was significantly higher in GFP-KCNQ2ΔE5 expressing cells (p<0.0001, ****). (FIG. 5G) The GFP signal intensity variation was significantly higher in GFP-KCNQ2ΔE5 expressing cells. (p<0.0001, ****). Statistical significance was determined by unpaired, two-tailed student's t-test (**** p<0.0001). Data are shown as mean±SEM, each circle corresponds to one cell.

FIGS. 6A-6H: Expression of KCNQ2^{ΔE5} causes intracellular accumulation and non-functional Kv7 channels. (FIG. 6A) Manual voltage clamp of CHO-K1 cells expressing hKCNQ2 transgenes in homozygous condition (KCNQ2^{WT}: n=9, KCNQ2^{ΔE5}: n=2); (left) Representative KCNQ2 current (XE-991-sensitive) traces for voltage-clamp protocol. (right) Summary data (mean±SEM) for outward current density as a function of command voltage. No observable current is observed when KCNQ2^{ΔE5} is expressed alone with KCNQ3. (FIG. 6B) Validation of GFP fused hKCNQ2 transgenes. (left) Schematic illustrating hKCNQ2 transgene and GFP fused construct. Image of CHO-K1 cells expressing GFP-fused lenti-transgene; (right) Summary data (mean±SEM) for outward current density as a function of command voltage. Fusion of GFP does not lead to significant alteration to KCNQ2 channel current. (FIG. 6C) Whole-cell voltage clamp recordings from CHO-K1 cells expressing GFP/RFP-fused hKCNQ2 transgenes in homozygous and heterozygous conditions; (left) Representative KCNQ2 current (XE-991-sensitive) traces for voltage-clamp protocol; (right) Summary data (mean±SEM) for outward current density as a function of command voltage (KCNQ2^{WT/WT}: n=6, KCNQ2^{ΔE5/WT}: n=4, KCNQ2^{ΔE5/ΔE5}: n=2). (FIG. 6D-FIG. 6E) Immunocytochemistry of GFP/RFP expressing CHO-K1 cells. Max intensity projections of three representative cells are shown for both conditions, GFP-WT+RFP-WT and GFP-KCNQ2^{ΔE5}+RFP+WT. Red dashed line outlines RFP-KCNQ2, and green dashed line outlines the same ROI for GFP channel. Cell body area as well as GFP and RFP signal metrics were extracted for each cell ROI (GFP-WT: n=19, KCNQ2^{ΔE5}: n=19). (FIG. 6F) Average cell body area was not significantly different between the two conditions. (FIG. 6G) Quantification of mean GFP signal was significantly higher in GFP-KCNQ2^{ΔE5}/RFP-WT expressing cells (p=0.0101). (FIG. 6H) Quantification of mean RFP signal was reduced in GFP-KCNQ2^{ΔE5}/RFP-WT expressing cells (p=0.0006). Statistical significance was determined by unpaired, two-tailed student's t-test (p<0.0001). Data are shown as mean±SEM, each circle corresponds to one cell.

FIGS. 7A-7J: KCNQ2^{ΔE5} forms aggregates in iPSC-derived neurons and postmortem ALS patient tissue. (FIG. 7A) Schematic illustration of CRISPR-Cas9 editing of human ESCs to generate homozygous KCNQ2^{ΔE5/ΔE5} lines. Isogenic control and KCNQ2^{ΔE5/ΔE5} ESCs were differentiated into cortical excitatory neurons for subsequent analysis (FIG. 7B) RT-PCR assay reveals KCNQ2 mutant cells only yield KCNQ2^{ΔE5} whereas controls only produce wildtype KCNQ2 in cortical neurons. (FIG. 7C) Representative ICC images of ESC-derived neurons stained for DAPI, MAP2, KCNQ2, and ANK-G. Arrow denotes beginning of axon initial segment (AIS). Plus (+), and minus (−) signs denote the presence or absence of KCNQ2 signal within the AIS. Yellow dashed line outlines neuronal cell body. Scale bar=10 mm. (FIG. 7D) Characterization of KCNQ2 expression pattern observed in neurons. KCNQ2 signal was characterized in the soma and AIS of each neuron and assigned into category. (FIG. 7E) Representative images of KCNQ2 signal within the neuronal cell body. Scale bar (right panel=10 mm, left images=2 mm) (FIG. 7F) Quantification of maximal GFP signal was significantly higher in KCNQ2^{ΔE5/ΔE5} neurons (p=0.0035). (FIG. 7G) KCNQ2 signal intensity variation was significantly higher in KCNQ2^{ΔE5/ΔE5} neurons (p<0.0001). Statistical significance was determined by unpaired, two-tailed student's t-test. Data are shown as mean±SEM, each circle corresponds to one neuron (control: n=51, KCNQ2^{ΔE5/ΔE5} n=79). (FIG. 7H) IHC of neurons from healthy control and sporadic ALS spinal cord samples stained for DAPI (blue), MAP2 (white), KCNQ2 (green), TDP-43 (red). (FIG. 7I) KCNQ2 signal intensity variation was measured from the cell body of neurons from three healthy controls and three sALS patients (represented in G). Each circle represents one neuron. (FIG. 7J) IHC of neurons from healthy control and sALS spinal cord samples stained for DAPI (blue), MAP2 (white), KCNQ2 (green), Ubiquitin (red). Square box denotes region magnified to the right of each representative image.

FIGS. 8A-8G: KCNQ2^{ΔE5} forms aggregates in iPSC-derived neurons and postmortem ALS patient tissue. (FIG. 8A) Details of CRISPR mutagenesis of KCNQ2. The four gRNAs targeting KCNQ2 are presented along with details of deletions induced in KCNQ2^{ΔE5/ΔE5} cells. (FIG. 8B) KCNQ2 allele copy number assay for unrelated iPSC cell line, isogenic control and KCNQ2^{ΔE5/ΔE5} ESCs. (FIG. 8C) Karyotype results for isogenic control and KCNQ2^{ΔE5/ΔE5} ESCs. (FIG. 8D) Representative images of NGN2 neurons stained with DAPI, GFP, MAP2, and KCNQ2. (FIG. 8E) Representative image and 3D views of KCNQ2^{ΔE5/ΔE5} neurons stained with DAPI, MAP2, Calnexin, and KCNQ2. Top panel shows 3D view when cell rotated forward, and bottom panel shows view of same neuron from below. (FIG. 8F) Related to FIG. 4H; representative images from healthy control and sporadic ALS spinal cord samples stained for DAPI (blue), MAP2 (white), KCNQ2 (green), TDP-43 (red). (FIG. 8G) Related to FIG. 4J; representative images from healthy control and sporadic ALS spinal cord samples stained for DAPI (blue), MAP2 (white), KCNQ2 (green), Ubiquitin (red).

FIGS. 9A-9B: ASO screen. (FIG. 9A) Schematic presentation of the screen pipeline. Newly designed and synthesized ASOs are screened using human spinal MNs treated with TDP-43 siRNAs with (w/) or without (w/o) KCNQ2 ASOs (3 uM). Mis-splicing of KCNQ2 is examined using RT-qPCR. (FIG. 9B) Splice-modulating ASOs can suppress the mis-splicing of KCNQ2. n=4 per condition, ****p<0.0001, **p<0.01, *p<0.05, unpaired t-test with Bonferroni correction, compared to NTC.

FIG. 10: Dose-dependent responses from two lead ASOs. Three doses of each ASO are included, 1, 3, and 10 uM. n=2-4 per condition, **** p<0.0001, One-way anova with Tukey's post hoc, compared to NTC.

FIGS. 11A-11B: Effects of KCNQ2 splice modulating ASOs on the expression of UNC13A and STMN2. n=2-4 per condition, ****p<0.0001, ***p<0.001, **p<0.01, One-way anova with Tukey's post hoc, compared to NTC.

FIGS. 12A-12B: Motor Neuron Electrophysiology. Knockdown of TDP-43 in motor neurons leads to (FIG. 12A) depolarization of the resting membrane potential and (FIG. 12B) increased spontaneous action potential firing. Treatment with splice-modulating KCNQ2 ASO (#2) in TDP-43 deficient motor neurons recovers RMP and spontaneous firing to control levels.

### DETAILED DESCRIPTION

The present disclosure addresses certain pathologies with associations between TDP-43 dysfunction and neurophysiology. By integrating gene expression datasets from human iPSC-derived neurons depleted of TDP-43 and postmortem ALS patient tissue the Applicants have identified spurious skipping of exon 5 of the voltage gated potassium channel KCNQ2 (Kv7.2) as a variant of interest (KCNQ2^{ΔE5}). KCNQ2 forms heterotetrameric Kv7 channels with other subunits, conducting M-currents and regulating repetitive firing and excitability in neurons, while rare missense variants in KCNQ2 cause a variety of epileptic syndromes. The Applicants have discovered that KCNQ2 is sensitive to TDP-43 levels and aberrant pre-mRNA processing yields a non-functional protein that accumulates within the ER and forms abundantly present ubiquitinated aggerates in patient tissue. Collectively, our findings highlight KCNQ2^{ΔE5} as a biomarker that can be leveraged for TDP-43 dysfunction, ALS and FTD-TDP diagnosis, and provide a functional connection between TDP-43 pathology and neuronal hyperexcitability in patients.

It is to be appreciated that certain aspects, modes, embodiments, variations and features of the present methods are described below in various levels of detail in order to provide a substantial understanding of the present technology. It is to be understood that the present disclosure is not limited to particular uses, methods, reagents, compounds, compositions or biological systems, which can, of course, vary. It is also to be understood that the terminology used herein is for the purpose of describing particular embodiments only, and is not intended to be limiting.

### Definitions

Unless defined otherwise, all technical and scientific terms used herein generally have the same meaning as commonly understood by one of ordinary skill in the art to which this technology belongs. As used in this specification and the appended claims, the singular forms “a”, “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

As used herein, the term “about” in reference to a number is generally taken to include numbers that fall within a range of 1%, 5%, or 10% in either direction (greater than or less than) of the number unless otherwise stated or otherwise evident from the context (except where such number would be less than 0% or exceed 100% of a possible value).

As used herein, the “administration” of an agent or drug to a subject includes any route of introducing or delivering to a subject a compound to perform its intended function. Administration can be carried out by any suitable route, including but not limited to, orally, intranasally, parenterally (intravenously, intramuscularly, intraperitoneally, or subcutaneously), rectally, intrathecally, intratumorally or topically. Administration includes self-administration and the administration by another.

As used herein, the term “effective amount” refers to a quantity sufficient to achieve a desired therapeutic and/or prophylactic effect, e.g., an amount which results in the prevention of, or a decrease in a disease or condition described herein or one or more signs or symptoms associated with a disease or condition described herein. In the context of therapeutic or prophylactic applications, the amount of a composition administered to the subject will vary depending on the composition, the degree, type, and severity of the disease and on the characteristics of the individual, such as general health, age, sex, body weight and tolerance to drugs. The skilled artisan will be able to determine appropriate dosages depending on these and other factors. The compositions can also be administered in combination with one or more additional therapeutic compounds. In the methods described herein, the therapeutic compositions may be administered to a subject having one or more signs or symptoms of a disease or condition described herein. As used herein, a “therapeutically effective amount” of a composition refers to composition levels in which the physiological effects of a disease or condition are ameliorated or eliminated. A therapeutically effective amount can be given in one or more administrations.

As used herein, the term “separate” therapeutic use refers to an administration of at least two active ingredients at the same time or at substantially the same time by different routes.

As used herein, the term “sequential” therapeutic use refers to administration of at least two active ingredients at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients. There is no simultaneous treatment in this case.

As used herein, the term “simultaneous” therapeutic use refers to the administration of at least two active ingredients by the same route and at the same time or at substantially the same time.

As used herein, the terms “subject”, “patient”, or “individual” can be an individual organism, a vertebrate, a mammal, or a human. In some embodiments, the subject, patient or individual is a human.

As used herein, the term “therapeutic agent” is intended to mean a compound that, when present in an effective amount, produces a desired therapeutic effect on a subject in need thereof.

“Treating” or “treatment” as used herein covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. In some embodiments, treatment means that the symptoms associated with the disease are, e.g., alleviated, reduced, cured, or placed in a state of remission.

In the context of the present disclosure, the following abbreviations for the commonly occurring nucleic acid bases are used. “A” refers to adenine, “C” refers to cytosine, “G” refers to guanine, “T” refers to thymine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “polynucleotide” as used herein is defined as a chain of nucleotides. Furthermore, nucleic acids are polymers of nucleotides. Thus, “nucleic acid” and “polynucleotide” as used herein are interchangeable. One skilled in the art has the general knowledge that nucleic acids are polynucleotides, which can be hydrolyzed into the monomeric “nucleotides” and which comprise one or more “nucleotide sequence(s)”. The monomeric nucleotides can be hydrolyzed into nucleosides. As used herein polynucleotides include, but are not limited to, all nucleic acid sequences (i.e., “nucleotide sequences”) which are obtained by any means available in the art, including, without limitation, recombinant means, i.e., the cloning of nucleic acid sequences from a recombinant library or a cell genome, using ordinary cloning technology and PCR, and the like, and by synthetic means.

As used herein, “expression” includes one or more of the following: transcription of the gene into precursor mRNA; splicing and other processing of the precursor mRNA to produce mature mRNA; mRNA stability; translation of the mature mRNA into protein (including codon usage and tRNA availability); and glycosylation and/or other modifications of the translation product, if required for proper expression and function.

A “fragment” is a portion of an amino acid sequence or a polynucleotide which is identical in sequence to but shorter in length than a reference sequence. A fragment may comprise up to the entire length of the reference sequence, minus at least one nucleotide/amino acid residue. For example, a fragment may comprise from 5 to 1000 contiguous nucleotides or contiguous amino acid residues of a reference polynucleotide or reference polypeptide, respectively. In some embodiments, a fragment may comprise at least 4, 5, 6, 7, 8, 9, 10, 11, 12, 13, 14, 15, or 16 contiguous amino acid residues of a reference peptide, respectively. Fragments may be preferentially selected from certain regions of a molecule. The term encompasses the full length polynucleotide or full length polypeptide.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same nucleobase or amino acid, then the molecules are homologous at that position. A degree of homology between sequences is a function of the number of matching or homologous positions shared by the sequences. A polynucleotide or polynucleotide region (or a polypeptide or polypeptide region) has a certain percentage (for example, at least 60%, 65%, 70%, 75%, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art. In some embodiments, default parameters are used for alignment. One alignment program is BLAST, using default parameters. In particular, programs are BLASTN and BLASTP, using the following default parameters: Genetic code=standard; filter-none; strand-both; cutoff=60; expect=10; Matrix=BLOSUM62; Descriptions=50 sequences; sort by=HIGH SCORE; Databases-non-redundant, GenBank+EMBL+DDBJ+PDB+GenBank CDS translations+SwissProtein+SPupdate+PIR. Details of these programs can be found at the National Center for Biotechnology Information. Biologically equivalent polynucleotides are those having the specified percent homology and encoding a polypeptide having the same or similar biological activity. Two sequences are deemed “unrelated” or “non-homologous” if they share less than 40% identity, or less than 25% identity, with each other.

As used herein, the term “gene” means a segment of DNA that contains all the information for the regulated biosynthesis of an RNA product, including promoters, exons, introns, and other untranslated regions that control expression.

As used herein, “oligonucleotide” refers to a molecule that has a sequence of nucleic acid bases on a backbone comprised mainly of identical monomer units at defined intervals. The bases are arranged on the backbone in such a way that they can bind with a nucleic acid having a sequence of bases that are complementary to the bases of the oligonucleotide. The most common oligonucleotides have a backbone of sugar phosphate units. A distinction may be made between oligodeoxyribonucleotides that do not have a hydroxyl group at the 2′ position and oligoribonucleotides that have a hydroxyl group at the 2′ position. Oligonucleotides may also include derivatives, in which the hydrogen of the hydroxyl group is replaced with organic groups, e.g., an allyl group. One or more bases of the oligonucleotide may also be modified to include a phosphorothioate bond (e.g., one of the two oxygen atoms in the phosphate backbone which is not involved in the internucleotide bridge, is replaced by a sulfur atom) to increase resistance to nuclease degradation. The exact size of the oligonucleotide will depend on many factors, which in turn depend on the ultimate function or use of the oligonucleotide. The oligonucleotide may be generated in any manner, including, for example, chemical synthesis, DNA replication, restriction endonuclease digestion of plasmids or phage DNA, reverse transcription, PCR, or a combination thereof. The oligonucleotide may be modified e.g., by addition of a methyl group, a biotin or digoxigenin moiety, a fluorescent tag or by using radioactive nucleotides.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein, the term “peptide” refers to a polymer of amino acid residues joined by amide linkages, which may optionally be chemically modified to achieve desired characteristics. The term “amino acid residue,” includes but is not limited to amino acid residues contained in the group consisting of alanine (Ala or A), cysteine (Cys or C), aspartic acid (Asp or D), glutamic acid (Glu or E), phenylalanine (Phe or F), glycine (Gly or G), histidine (His or H), isoleucine (Ile or I), lysine (Lys or K), leucine (Leu or L), methionine (Met or M), asparagine (Asn or N), proline (Pro or P), glutamine (Gln or Q), arginine (Arg or R), serine (Ser or S), threonine (Thr or T), valine (Val or V), tryptophan (Trp or W), and tyrosine (Tyr or Y) residues. The term “amino acid residue” also may include unnatural amino acids or residues contained in the group consisting of homocysteine, 2-Aminoadipic acid, N-Ethylasparagine, 3-Aminoadipic acid, Hydroxylysine, β-alanine, β-Amino-propionic acid, allo-Hydroxylysine acid, 2-Aminobutyric acid, 3-Hydroxyproline, 4-Aminobutyric acid, 4-Hydroxyproline, piperidinic acid, 6-Aminocaproic acid, Isodesmosine, 2-Aminoheptanoic acid, allo-Isoleucine, 2-Aminoisobutyric acid, N-Methylglycine, sarcosine, 3-Aminoisobutyric acid, N-Methylisoleucine, 2-Aminopimelic acid, 6-N-Methyllysine, 2,4-Diaminobutyric acid, N-Methylvaline, Desmosine, Norvaline, 2,2′-Diaminopimelic acid, Norleucine, 2,3-Diaminopropionic acid, Ornithine, and N-Ethylglycine. Typically, the amide linkages of the peptides are formed from an amino group of the backbone of one amino acid and a carboxyl group of the backbone of another amino acid.

A “target site” or “target sequence” refers to a nucleic acid sequence that defines a portion of a nucleic acid to which a binding molecule may specifically bind under conditions sufficient for binding to occur. In some embodiments, a target sequence refers to a genomic nucleic acid sequence that defines a portion of a nucleic acid to which a binding molecule may specifically bind under conditions sufficient for binding to occur.

It is also to be appreciated that the various modes of treatment of disorders as described herein are intended to mean “substantial,” which includes total but also less than total treatment, and wherein some biologically or medically relevant result is achieved. The treatment may be a continuous prolonged treatment for a chronic disease or a single, or few time administrations for the treatment of an acute condition.

### Antisense Oligonucleotides

In one aspect, the present disclosure provides an antisense oligonucleotide (ASO), comprising, consisting of, or consisting essentially of 10 to 30 nucleotides. For example, the ASO may comprise, consist or, or consist essentially of 10, 11, 12, 13, 14, 15, 16, 17, 18, 19, 20, 21, 22, 23, 24, 25, 26, 27, 28, 29, or 30 nucleotides. The ASO binds to a target region comprising, consisting of, or consisting essentially of an intronic sequence between exons 4 and 6 of a nucleic acid molecule encoding a human KCNQ2 protein. In some aspects, the ASO is modified or unmodified

In one aspects, the target region comprises, consists of, or consists essentially of a nucleic acid sequence of:

(SEQ ID NO: 19)
TGGTCATTGTGGTTTTCCCTTTCCCTGCTGATACACCCCTGTCCCTGTG
CTGGGACCAGGCTCTCACTGGCTGAGCCTGCTCCATACATCTCTTTGGG
GCCACCTGCTGGCTGCCCGTGGTCATGATGGCTTGTGGTCGAGGCGGGG
TGGTGGTTGCCAAGCTGGCGGGGGAGTGGGTGGGGAGGGCTGGGCCCCA
GAGAGCCTGAGCTCAGCCGTGGTGGGCATCTTCCTTCCTCCTGAGCTCA
GCCGTGGTGGGCATCTTCCTTCCTCCTGAGTGACCCTCCACCTGCTCCG
TGTCCCGATTATTGGGG
or
(SEQ ID NO: 20)
TGGTGGTGGTGGTGATGGTCATGATGGTCATGGTGGTGGTGGTGGTGAT
GGTGATGGTGGTGATCGTGGTGGTGGTGATGGTGATGGTGGTGGTGATG
ATGGTGGTGATGGTGGTGATGGTGATGGTGGTGGTGATGGTGATGGTGA
T.

ASO that bind to the target sequences may have a nucleic acid sequence that comprises or consists of CACCATCACCATCACCACCA (SEQ ID NO: 21), ACCATCACCATCACCACCA (SEQ ID NO: 22), ACCATCACCACCACCACCA (SEQ ID NO: 24), ATCACCATCACCATCACCA (SEQ ID NO: 25), CACCACCATCATCACCAC (SEQ ID NO: 33), CACCATCACCATCACCA (SEQ ID NO: 26), ATCACCATCACCATCA (SEQ ID NO: 27), AGGGAAAACCACAATGACCA (SEQ ID NO: 28), CCATCATGACCACGGGCAGCC (SEQ ID NO: 29), ATGACCACGGGCAGCCAGCA (SEQ ID NO: 30), ACCACGGCTGAGCTCAGGCTCT (SEQ ID NO: 31), or TCACTCAGGAGGAAGGAAGAT (SEQ ID NO: 32). In other words, the disclosed ASO may comprise or consist of a nucleic acid sequence of CACCATCACCATCACCACCA (SEQ ID NO: 21) or a pharmaceutically acceptable salt thereof; ACCATCACCATCACCACCA (SEQ ID NO: 22) or a pharmaceutically acceptable salt thereof; ATCACCATCACCACCACCA (SEQ ID NO: 23) or a pharmaceutically acceptable salt thereof; ACCATCACCACCACCACCA (SEQ ID NO: 24) or a pharmaceutically acceptable salt thereof, ATCACCATCACCATCACCA (SEQ ID NO: 25) or a pharmaceutically acceptable salt thereof; CACCACCATCATCACCAC (SEQ ID NO: 33) or a pharmaceutically acceptable salt thereof; CACCATCACCATCACCA (SEQ ID NO: 26) or a pharmaceutically acceptable salt thereof; ATCACCATCACCATCA (SEQ ID NO: 27) or a pharmaceutically acceptable salt thereof; AGGGAAAACCACAATGACCA (SEQ ID NO: 28) or a pharmaceutically acceptable salt thereof; CCATCATGACCACGGGCAGCC (SEQ ID NO: 29) or a pharmaceutically acceptable salt thereof; ATGACCACGGGCAGCCAGCA (SEQ ID NO: 30) or a pharmaceutically acceptable salt thereof; ACCACGGCTGAGCTCAGGCTCT (SEQ ID NO: 31) or a pharmaceutically acceptable salt thereof; or TCACTCAGGAGGAAGGAAGAT (SEQ ID NO: 32) or a pharmaceutically acceptable salt thereof.

The disclosed ASO may comprise at least one modified internucleoside linkage (i.e., an internucleoside linkage that is not a phosphodiester). Modified internucleoside linkages that can be incorporated into the disclosed ASO include, but are not limited to, phosphorothioate linkages (PS), cyclic guanidine phosphoramidate linkages (PG), and mesyl phosphoramidate linkages (MS). In some embodiments, the disclosed ASO may comprise 1, 2, 3, 4, 5, 6, 7, 8, 9, 10, 11, 12, 13, 14, 15, 16, 17, 18, 19, or 20 or more modified internucleoside linkages. In some embodiments, the disclosed ASO may comprise 1, 2, 3, 4, 5, 6, 7, 8, 9, 10, 11, 12, 13, 14, 15, 16, 17, 18, 19, or 20 or more phosphorothioate linkages. In some embodiments, all of the internucleoside linkages may be modified internucleoside linkages. In some embodiments, all of the internucleoside linkages may be phosphorothioate linkages.

Additionally or alternatively, the disclosed ASO may comprise at least one modified nucleotide. Modified nucleotides that can be incorporated into ASO are known in the art and include, but are not limited to, 2′-O-methoxyethyl (MOE) nucleotides, 2′-O-methyl (OMe) nucleotides, and 2′-fluoro (F) nucleotides. In some embodiments, the disclosed ASO may comprise 1, 2, 3, 4, 5, 6, 7, 8, 9, 10, 11, 12, 13, 14, 15, 16, 17, 18, 19, or 20 or more modified nucleotides. In some embodiments, the disclosed ASO may comprise 1, 2, 3, 4, 5, 6, 7, 8, 9, 10, 11, 12, 13, 14, 15, 16, 17, 18, 19, or 20 or more 2′-MOE nucleotides. In some embodiments, all of the nucleotides in the ASO may be modified nucleotides. In some embodiments, all of the nucleotides in the ASO may be 2′-MOE nucleotides.

In some aspects, the disclosed ASO comprise or consist of a nucleic acid sequence selected from CACCATCACCATCACCACCA (SEQ ID NO: 21), ACCATCACCATCACCACCA (SEQ ID NO: 22), ACCATCACCACCACCACCA (SEQ ID NO: 24), ATCACCATCACCATCACCA (SEQ ID NO: 25), CACCACCATCATCACCAC (SEQ ID NO: 33), CACCATCACCATCACCA (SEQ ID NO: 26), ATCACCATCACCATCA (SEQ ID NO: 27), AGGGAAAACCACAATGACCA (SEQ ID NO: 28), CCATCATGACCACGGGCAGCC (SEQ ID NO: 29), ATGACCACGGGCAGCCAGCA (SEQ ID NO: 30), ACCACGGCTGAGCTCAGGCTCT (SEQ ID NO: 31), and TCACTCAGGAGGAAGGAAGAT (SEQ ID NO: 32); wherein all of the internucleoside linkages connecting the nucleotides are phosphorothioate linkages, and all of the nucleotides are 2′-MOE nucleotides.

### Treatments

The disclosed ASO can prevent mis-splicing of KCNQ2, and therefore can regulate and correct TDP-43 cytoplasmic aggregation, TDP-43 nuclear depletion, or both. As noted throughout, -43 cytoplasmic aggregation and nuclear depletion are common hallmarks of certain neurodegenerative diseases including Amyotrophic Lateral Sclerosis (ALS) and frontotemporal dementia (FTD). The disclosed ASO can be used to treat such conditions.

Thus, at a high level, the present disclosure provides method of preventing KCNQ2 mis-splicing in a cell, comprising contacting the cell with the ASO or the pharmaceutical composition thereof.

Further, the present disclosure provides methods of treating or preventing a neurodegenerative disease in a subject, comprising administering any one (or more) of the disclosed ASO or a pharmaceutical composition thereof to a subject with a neurodegenerative disease. In some embodiments, the neurodegenerative disease involves TDP-43 cytoplasmic aggregation, TDP-43 nuclear depletion, or both. In some embodiments, the neurodegenerative disease is Amyotrophic Lateral Sclerosis (ALS) or frontotemporal dementia (FTD). In some embodiments, the subject is a human.

### EXAMPLES

The present technology is further illustrated by the following Examples, which should not be construed as limiting in any way.

The following proposed experiments involve a series of technically innovative methods, including the production of cortical neurons from human iPSCs, the design of splice-modulating ASOs, and the generation of a humanized KCNQ2 knock-in mouse model. The work is conceptually innovative in that it will determine the functional role of KCNQ2 in human neurons and assess the therapeutic potential of restoring loss-of-function of KCNQ2 using human iPSC and humanized mouse models.

The Applicants sought to address any potential association between TDP-43 dysfunction and neurophysiology. By integrating gene expression datasets from human iPSC-derived neurons depleted of TDP-43 and postmortem ALS patient tissue the Applicants have identified spurious skipping of exon 5 of the voltage gated potassium channel KCNQ2 (Kv7.2) as a variant of interest (KCNQ2^{ΔE5}). KCNQ2 forms heterotetrameric Kv7 channels with other subunits, conducting M-currents and regulating repetitive firing and excitability in neurons, while rare missense variants in KCNQ2 cause a variety of epileptic syndromes²⁴. The Applicants found that KCNQ2 is sensitive to TDP-43 levels and aberrant pre-mRNA processing yields a non-functional protein that accumulates within the ER and forms abundantly present ubiquitinated aggerates in patient tissue. Collectively, our findings highlight KCNQ2^{ΔE5} as a biomarker that can be leveraged for TDP-43 dysfunction, ALS and FTD-TDP diagnosis, and provide a functional connection between TDP-43 pathology and neuronal hyperexcitability in patients.

The Applicants demonstrate that the voltage-gated potassium channel KCNQ2 is spuriously mis-spliced in the context of ALS. Using iPSC-derived neurons and postmortem brain tissue the Applicants found widespread, yet highly ALS-specific skipping of a constitutive exon encoding the K⁺ pore domain. Activation of this event is triggered by TDP-43 loss-of-function and correlates with phosphorylated TDP-43 levels and age of disease onset in patients. The mis-spliced KCNQ2 channel is incapable of conducting ions but does not exhibit dominant negative activity. It forms cytoplasmic, ubiquitinated aggregates in differentiated neurons and patient samples and can be leveraged as a novel biomarker for TDP-43 pathology and ALS diagnosis. Our work reveals that TDP-43 maintains the fidelity of KCNQ2 expression and function in human neurons and provides a mechanistic link between established excitability disturbances in ALS patients and TDP-43 dysfunction.

### Example 1: Materials and Methods

### Results

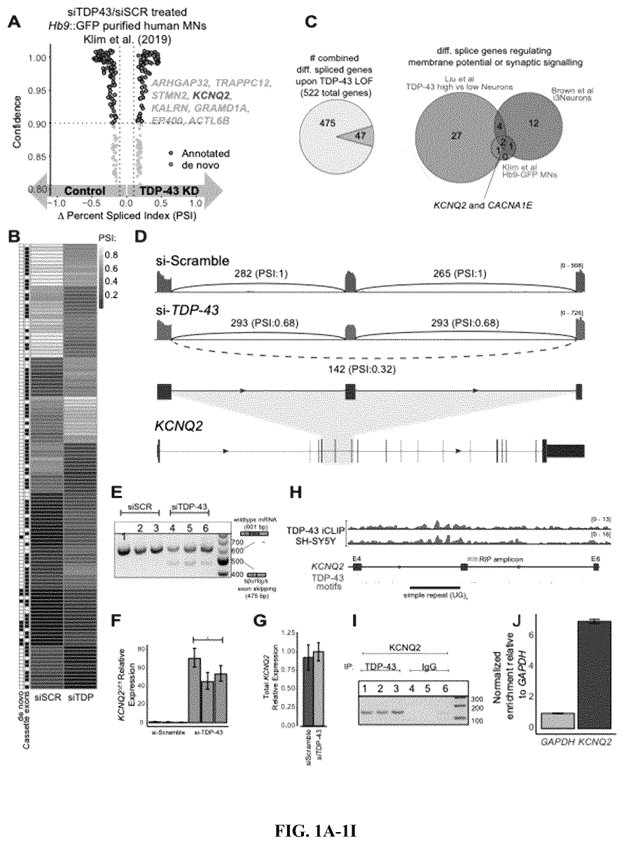

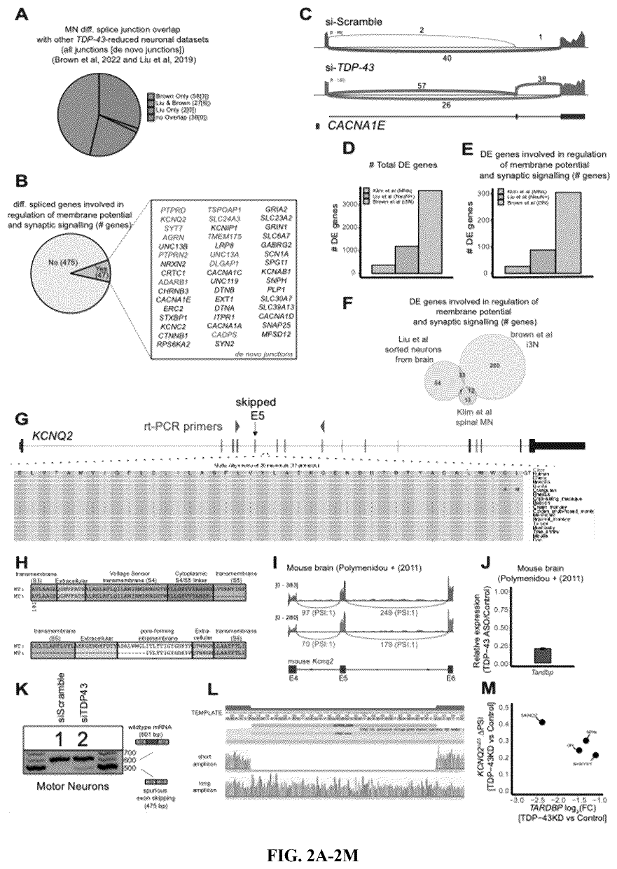

Purified spinal iPSC-MNs exhibit several TDP-43 dependent splicing events. An RNA sequencing (RNA-seq) dataset was analyzed using the MAJIQ pipeline and identified a stringent set of 125 differential splicing variants including the potassium voltage-gated ion channel KCNQ2 (supported by |Δ Percent Select Index [ΔPSI]|>0.15 and Probability of change>0.8) (FIG. 1A). Classification of simple and complex splice variants revealed that a majority of TDP-43 sensitive events involve cassette exons (FIG. 1B). Since TDP-43 is known to suppress the inclusion of cryptic exons, the Applicants surveyed for such events within our results. Of note, the Applicants discovered 9 unique and statistically significant de novo processing events that are neither detected in control-treated neurons, nor defined within current gene annotations, including premature termination events in STMN2 and ARHGAP32, cryptic exon inclusions in KALRN and others (FIG. 1A). The Applicants next asked if the splice junctions the Applicants identified in MNs were prevalent across other datasets of TDP-43 dysfunction recently reported in iPSC-derived cortical neurons and neuronal nuclei from ALS/FTD postmortem brains (FIG. 2A). Despite using different criteria for defining differential splicing, Applicants observed significant overlap, with 87/125 of MN events also detected in additional datasets (FIG. 2A). This was not only restricted to alternative splicing events, but also de novo or cryptic splicing events (FIG. 2A). These analyses add further clarity on TDP-43 regulated processes in distinct neuronal cells and provide a framework to investigate the mechanistic interaction between TDP-43 loss-of-function and disease processes.

TDP-43 is required for the inclusion of a deeply conserved constitutive exon in KCNQ2. The identification of KCNQ2 as a differentially spliced gene in MNs (FIG. 1A) suggested a potential connection between TDP-43 dysfunction and neuronal excitability. To investigate this connection further, Applicants integrated splicing changes from all three datasets interrogating TDP-43-depletion (iPSC-derived MNs⁹, iPSC-cortical neurons⁷ and ALS/FTD neuronal nuclei) to arrive at 522 significantly differentially spliced genes (FIG. 1C, left and FIG. 2B, left). Consistent with our view, this stringent set was enriched for genes with connections to human phenotypes such as motor seizures, abnormal motor function and related terms. Intersecting differentially spliced genes with GO terms such as “regulation of membrane potential” and “synaptic signaling” highlighted 47 target genes (FIG. 1C, right and FIG. 2B, right), only 2 of which were commonly affected across all 3 datasets, KCNQ2 and the calcium voltage-gated channel CACNA1E (FIG. 1B, right). A similar analysis integrating gene expression did not yield common targets dysregulated in all three datasets (FIG. 2D-F), so Applicants focused instead on splicing defects. While the TDP-43 dependent event on CACNA1E represents the preferential splicing of a known, annotated isoform (FIG. 2C), loss of TDP-43 results in the skipping of exon 5 in the KCNQ2 transcript (FIG. 1D). Notably, this is not an annotated cassette exon, and Applicant was unable to detect reads supporting this processing event in control datasets, as well as diverse human tissue. The unexpectedly skipped exon codes for a deeply conserved segment (FIG. 2G) of the pore domain that conducts K⁺ (FIG. 2H), suggesting that this event could have deleterious effects on KCNQ2 expression and function. Skipping of exon 5 is not detected in published TDP-43 mouse models (FIG. 2I-J), suggesting that this splicing event may bear similarities to other human-specific targets such as STMN2 and UNC13A. Thus, Applicants categorize this alteration as an aberrant splicing event and refer to the resulting exon 5 skipped transcript as KCNQ2^{ΔE5}.

Applicants used PCR coupled to Sanger sequencing to validate the presence of KCNQ2^{ΔE5} in iPSC-derived excitatory neurons (FIGS. 1E-F) and MNs (FIGS. 2K-L) that were depleted of TDP-43. Notably, the product corresponding to exon 5 skipping was detectable almost exclusively in neurons treated with an siRNA against TDP-43, but not in controls treated with an siRNA scrambled sequence (si-TDP-43 and siScramble respectively). KCNQ2^{ΔE5} is also detectable in published RNA-seq datasets generated using SH-SY5Y and SK-N-DZ neuronal lines, as well as i³ cortical neurons, suggesting this target may be highly sensitive to TDP-43 levels (FIG. 2M). Other recently discovered human neuronal TDP-43 targets like STMN2 and UNC13A undergo cryptic exon inclusion that results in decrease of the host gene either through premature transcription termination or nonsense mediated decay (NMD). However, skipping of exon 5 in KCNQ2 does not alter the coding frame and analysis by quantitative RT-PCR showed that the overall expression levels of KCNQ2 in TDP-43 depleted neurons remain unchanged, despite the dramatic mis-splicing into KCNQ2^{ΔE5} (FIG. 1G).

TDP-43 is known to bind UG repeats within long introns. To explore whether TDP-43 may directly associate with KCNQ2 mRNA Applicants first examined sequence content surrounding the skipped exon 5. While there are many UG dinucleotides within the 5 kb locus containing exon 5 and flanking introns, Applicants observed an annotated ~1 kb long, simple repeat 6 bp upstream of exon 5 (FIG. 1H). Remarkably, this region consists of UG repeats, suggesting the potential for direct TDP-43 recruitment. Mapping of published TDP-43 individual cross-linking and immunoprecipitation (iCLIP) datasets in neuroblastoma SH-SY5Y cells to this locus showed broad TDP-43 binding within KCNQ2, including upstream of exon 5 (FIG. 1H). To validate this CLIP enrichment, Applicants UV-crosslinked SH-SY5Y cells and utilized a protocol to immunoprecipitate (IP) TDP-43 and associated RNA targets. In support of our hypothesis, Applicants detected specific and TDP43-IP-dependent KCNQ2 pre-mRNA enrichment (FIG. 1I-J). Altogether, Applicants demonstrated that TDP-43 has a critical and human-specific role in ensuring the integrity of KCNQ2 processing.

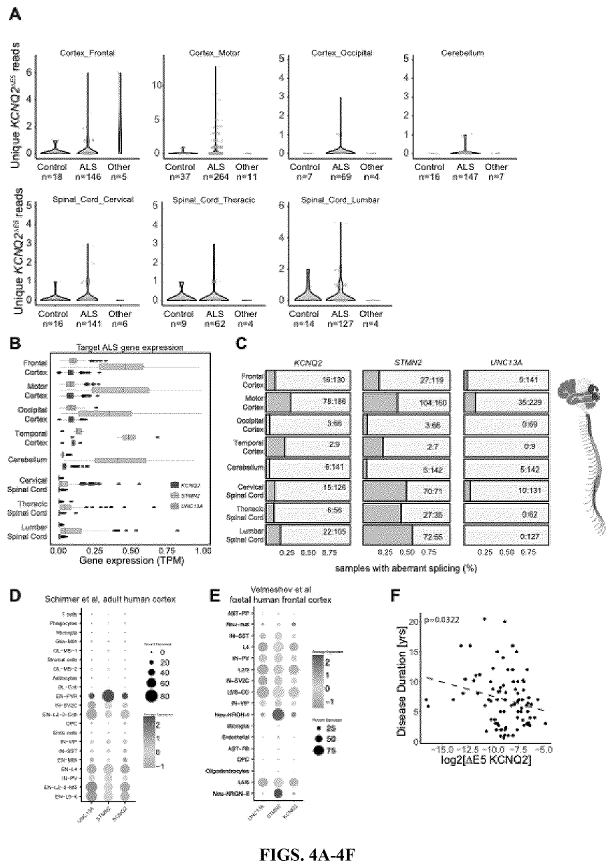

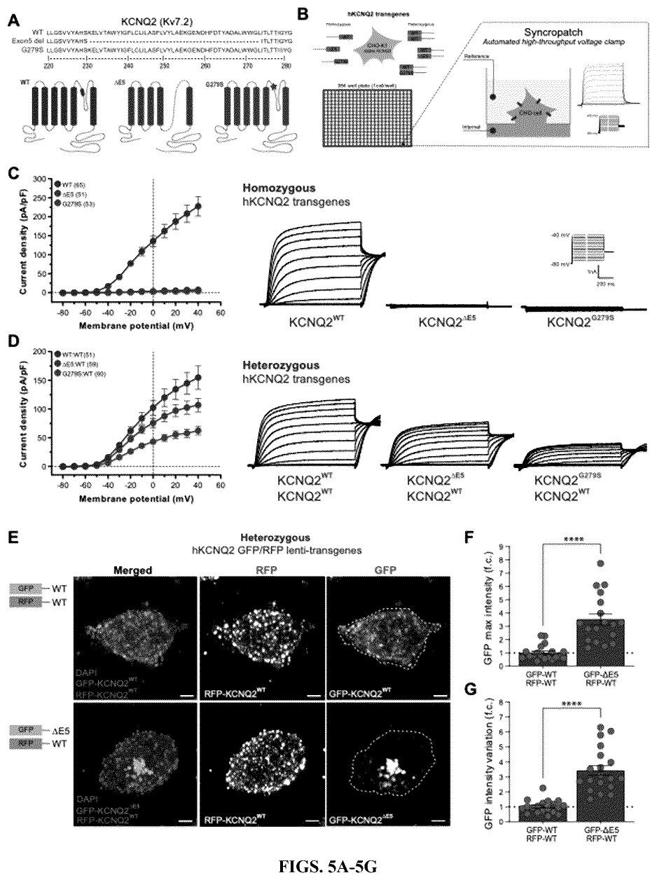

TDP-43 dependent processing of KCNQ2 is altered in postmortem ALS/FTD CNS tissue. The identification of KCNQ2 mis-splicing in iPSC-derived neurons depleted of TDP-43 prompted us to investigate the extent of this event in ALS patient postmortem tissue. Applicants first examined RNA sequencing datasets generated by the Target ALS Human Postmortem Tissue Core at the New York Genome Center, consisting of 1272 libraries from diverse tissues of ALS patients (216), non-neurological disease controls (28), and other neurological disease individuals (8) (FIG. 2A). To evaluate the state of KCNQ2 processing, Applicants counted sequencing reads that stringently aligned to the KCNQ2 exon 4/6 boundary and thus corresponds to the aberrant KCNQ2^{ΔE5} mRNA. (FIG. 2B). This analysis revealed that KCNQ2 misprocessing was detectable and highly specific to the ALS cohort (FIG. 2C). The misprocessed KCNQ2^{ΔE5} mRNA was detected in most CNS tissues including different regions of the spinal cord, as well as in temporal, frontal and motor cortex within the brain (FIG. 2D). Notably, as much as 29.5% of motor cortex and 17.3% of lumbar spinal cord samples exhibited KCNQ2^{ΔE5}, in contrast to only 4.1% and 4.3% in the cerebellum and occipital cortex respectively (FIGS. 2D and 4A). While these tissue-specific differences in detection of KCNQ2^{ΔE5} could be reflecting relative disease burden and TDP-43 pathology, they are also likely associated with the relative expression level of KCNQ2 within the CNS, where it is appears more abundant in the brain than the spinal cord (FIG. 4B). Comparison of aberrant KCNQ2 levels with other known TDP-43 targets like STMN2 and UNC13A further revealed differential detection of aberrantly spliced products, with STMN2 misprocessing prominent within the spinal cord and motor cortex, and UNC13A within the motor cortex (FIG. 4C). For these genes as well, differential levels of mis-splicing across tissues are not strictly driven by their relative abundance (FIG. 4B-E).

As KCNQ2 splicing is dependent on TDP-43 functional activity in iPSC-derived neurons, Applicants next evaluated this correlation in postmortem patient tissue in vivo. Cytoplasmic TDP-43 aggregation is observed in an overwhelming majority of ALS patients (>97%) and is typically accompanied by nuclear loss-of-function. Thus, to interrogate whether detection of KCNQ2^{ΔE5} was related to TDP-43 status, Applicants grouped ALS datasets into those known to have typical cytoplasmic TDP-43 neuropathology (C9orf72 and sporadic cases) and those without (patients with SOD1 mutations). Applicants found that KCNQ2^{ΔE5} was only observed in patient samples expected to exhibit TDP-43 aggregation (FIG. 2E). To provide direct evidence for this relationship, Applicants analyzed RNA from a cohort of 89 frontal cortex brain from patients diagnosed with FTD and 27 healthy controls from the Mayo Clinic Brain Bank. These samples were characterized for TDP-43 pathology by immunohistochemistry and had documented clinical history. Using quantitative RT-PCR primers that distinguish wildtype and KCNQ2^{ΔE5} Applicants found a highly significant difference, with KCNQ2^{ΔE5} detected exclusively in patient samples, while as expected the levels of overall KCNQ2 transcript were not different (FIG. 2F-G). Critically, the level of misprocessed KCNQ2^{ΔE5} was directly and significantly associated with the level of phosphorylated TDP-43 levels (FIG. 2H), while there was an inverse relationship between the age of disease onset as well as disease duration and the abundance of KCNQ2^{ΔE5} in these patients (FIGS. 2I and 4F), suggesting a potential involvement of KCNQ2 dysfunction with disease mechanisms.

Lastly, to assess the extent of KCNQ2 mis-splicing within individual patient neurons with TDP-43 dysfunction, Applicants resorted to the dataset reported by Liu et al., in which neuronal nuclei from postmortem brains of ALS/FTD individuals were purified by fluorescence-activated cell sorting (FACS) based on neuronal nuclear protein (NeuN) expression and TDP-43 abundance and profiled by RNA-seq. While all the data are from ALS/FTD patients with C9orf72 expansions, Applicants compared the transcriptomes of neuronal nuclei with high and low TDP-43 levels. Strikingly, for six of six individuals, Applicants exclusively observed reads indicative of KCNQ2^{ΔE5} in neuronal nuclei with low TDP-43 levels (FIG. 2K-J). The level of disruption in splicing amounted to ~50% of total host gene expression, suggesting that if KCNQ2^{ΔE5} encodes a dysfunctional protein, it could reduce half of the total KCNQ2 available to neurons (FIG. 2K-J). Collectively, these analyses demonstrate that KCNQ2^{ΔE5} is a molecular biomarker of TDP-43 ALS/FTD pathology.

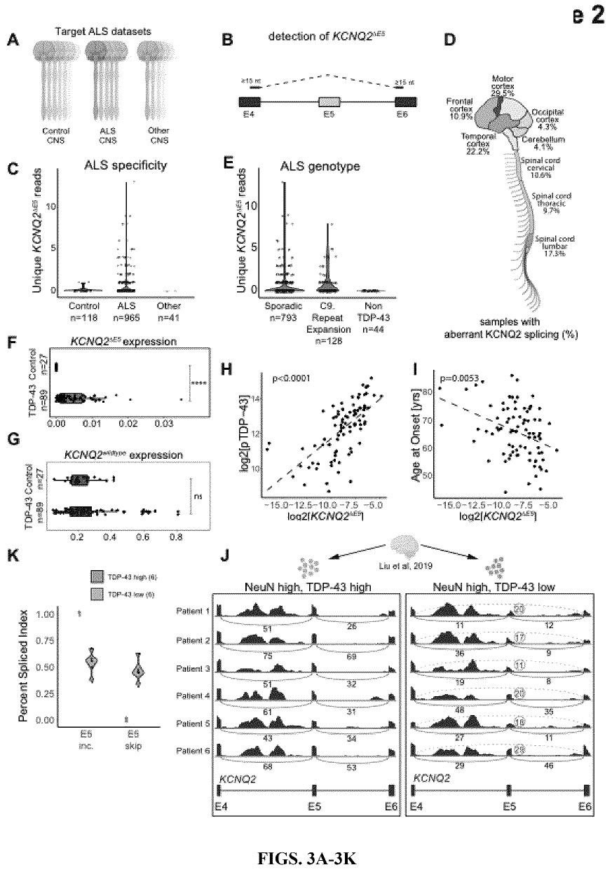

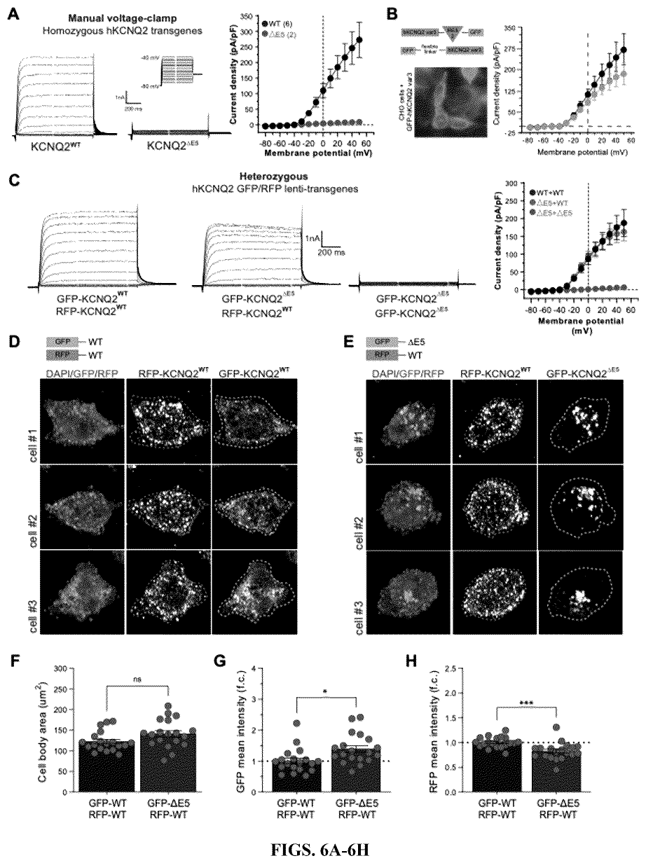

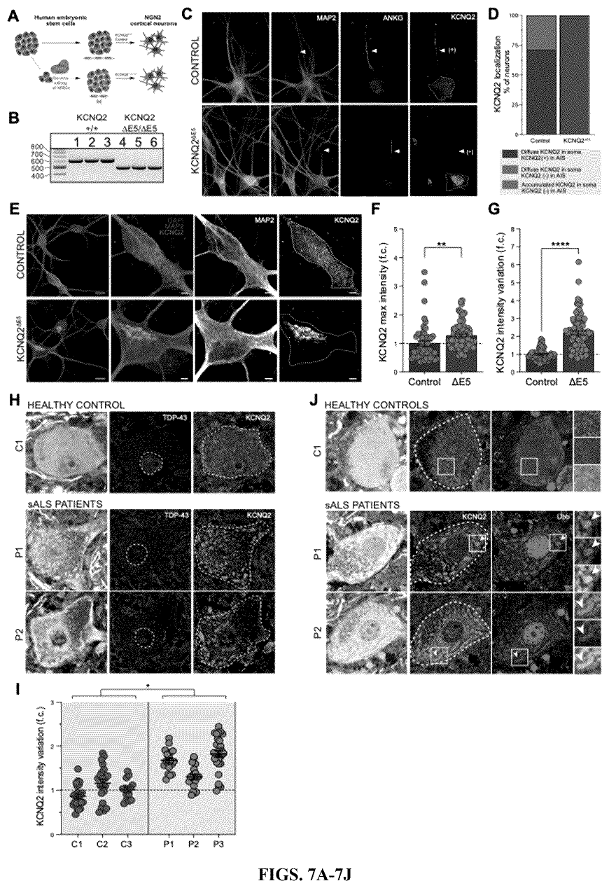

KCNQ2^{ΔE5} is a loss of function allele. Having established that KCNQ2^{ΔE5} is induced by TDP-43 loss of function in iPSC-derived neurons and ALS/FTD patient tissue, Applicants next wondered how mis-splicing of this gene might affect the biology of the encoded protein. The KCNQ2 gene encodes a voltage-gated K⁺ channel (Kv7.2) subunit that is expressed very early in the developing brain and forms homo-tetrameric and hetero-tetrameric K⁺ ion channels in combination with KCNQ3. The resulting voltage-gated potassium channels underlie the repolarizing M-current, which limits repetitive firing during long-lasting depolarizing inputs. Heterozygous mutations in KCNQ2 can cause a range of epileptic syndromes including benign self-limiting familial neonatal epilepsy (SLFNE; associated with haploinsufficiency mutations), to sporadic cases of severe developmental and epileptic encephalopathy (DEE; associated with dominant-negative loss-of-function mutations) that are characterized by neonatal seizures and a spectrum of cognitive, developmental, and motor deficits. To interrogate the function of the KCNQ2^{ΔE5} encoded protein Applicants generated a mutant expression plasmid lacking the corresponding exon 5 that is predicted to disrupt the K⁺ pore (FIG. 3A). Applicants transfected this plasmid along with a wildtype version in a CHO cell line that stably expresses KCNQ3 and performed manual patch clamp to measure K⁺ current density across a series of voltage steps. Notably, as CHO cells do not express any other ion channels, any voltage dependent current flow measurements will reflect the activity of the transfected KCNQ2/KCNQ3 tetramers. Applicants found that in stark contrast to the wildtype, KCNQ2^{ΔE5} expressing cells exhibited no current (FIG. 6A). To substantiate this observation across a larger cohort of cells (n>51) Applicants used the automated high throughput patch clamp SynchroPatch system (FIG. 3B). As an additional control Applicants included a construct expressing a recurrent KCNQ2 DEE-associated mutation known to exert a dominant negative effect (KCNQ2^{G279S}) and interrogated channel function at the homozygous state (FIG. 3C) and heterozygous state (FIG. 3D). These experiments clearly showed that the TDP-43 dependent exon skipping event on KCNQ2 renders the channel incapable of conducting any current (FIG. 3C). Importantly however, Applicants found no evidence for dominant negative activity, as KCNQ2^{ΔE5} did not impair the activity of the co-expressed wildtype channel (FIG. 3D). This contrasted with the KCNQ2^{G279S} variant, which as expected resulted in 65% reduction in current density in the heterozygous state (FIG. 3D).

Structure prediction suggests that the exon skipping event in KCNQ2 disrupts the formation of a functional pore domain, which may explain the inability to conduct current. However, it is also possible that the aberrant KCNQ2^{ΔE5} protein does express stably or traffic properly to the membrane. To address these possibilities, Applicants generated a series of wildtype and KCNQ2^{ΔE5} expression constructs fused with either GFP or RFP and as before transfected these in KCNQ3-expressing cells. Applicants first confirmed that the fusion did not impede the functional capacity of the wildtype channel (FIG. 6B) and that the KCNQ2^{ΔE5} exhibited the same reduction in current density measurements in the heterozygous state (FIG. 6C). Applicants then used immunocytochemistry and confocal microscopy to interrogate the expression and localization of KCNQ2. Intriguingly, Applicants found that while there was no significant difference in cell body between the wildtype and ΔE5-expressing cells (FIG. 6F), co-transfection caused a moderate but significant imbalance with higher levels of mutant and contaminant lower level of the wildtype protein (FIG. 6G-H). Most importantly, these experiments revealed a dramatic shift in the distribution of the proteins with the majority of the KCNQ2^{ΔE5}-GFP signal accumulating focally within the cell body (FIG. 3E and FIG. 6D-E). This observation was reflected by the significant increase in max intensity and standard deviation of the KCNQ2^{ΔE5}-GFP signal relative to the wildtype KCNQ2-GFP protein (FIG. 3F), suggesting that inefficient trafficking contributes to the loss-of-function effects of TDP-43 dysfunction on KCNQ2.

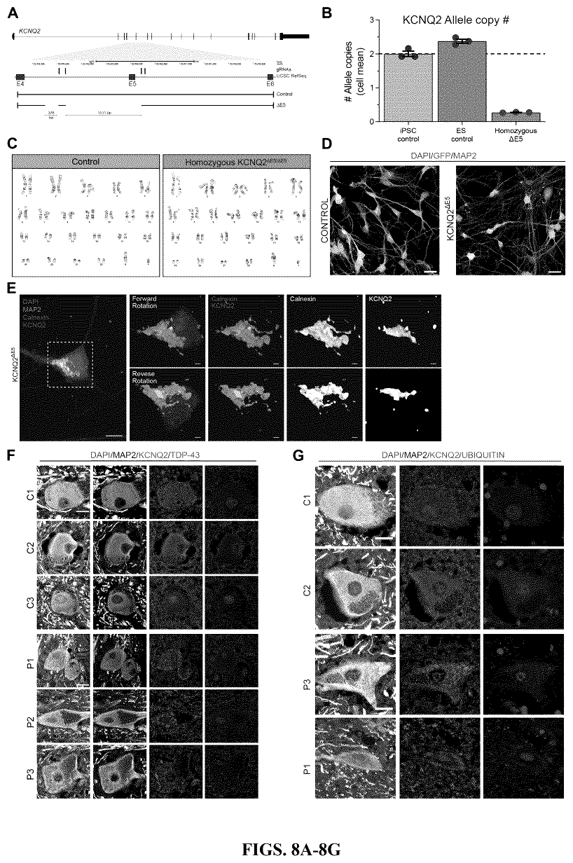

KCNQ2^{ΔE5} forms aggregates in iPSC-derived neurons and postmortem ALS patient tissue. Applicants next sought to address the relevance of these findings in the context of human neurons. To interrogate the homeostasis of KCNQ2^{ΔE5} in the absence of all the additional effects of TDP-43 dysfunction Applicants used CRISPR/Cas9 genome engineering to delete exon 5 in a healthy control human embryonic stem cell (ESC) line (FIG. 4A and FIG. 8A). Applicants generated a clone with a homozygous deletion in KCNQ2 exon 5 and confirmed that it had a normal karyotype (FIG. 8B) and no other on-target insertions by performing a quantitative genomic DNA PCR assay to assess allele copy number (FIG. 8C). Applicants then differentiated the KCNQ2 mutant and parental isogenic control cell lines into cortical excitatory neurons (FIG. 8D) and performed immunocytochemistry with a KCNQ2 antibody to assess the localization of the endogenous protein after 50-60 days in culture. To earmark the axon initial segment (AIS), which is the site where KCNQ2 channels localize in mature neurons Applicants used an antibody for ankyrin-G (FIG. 4B). Applicants found that 75% of wildtype neurons exhibited a diffuse staining pattern for KCNQ2 with a distinct signal within the AIS, that was in stark contrast to the KCNQ2^{ΔE5}-derived neurons, 100% of which exhibited focal intracellular accumulation of KCNQ2 within the cell body (FIG. 4B-D). Quantification of the KCNQ2 signal distribution revealed a significant increase in max intensity and standard deviation in mutant neurons relative to isogenic controls, essentially phenocopying the findings from the heterologous expression cell models (FIG. 4D-E). The dramatic focal accumulation of mis-spliced protein prompted us to determine the precise site of localization. Immunostaining with the ER marker Calnexin revealed substantial overlap, suggesting that the majority of KCNQ24E5 protein gets stuck within the ER in neurons and does not traffic to the AIS and neuronal membrane (FIG. 8E). Lastly, Applicants used sporadic ALS patient spinal cord sections to investigate the localization of KCNQ2 protein in vivo. Applicants performed immunohistochemistry in postmortem tissue from 3 cases and 3 controls (Table 1) to characterize TDP-43 and KCNQ2. Applicants found that in MAP2 positive large motor neurons from healthy control individuals, KCNQ2 exhibited a broad diffuse pattern throughout the cell body and clear nuclear TDP-43 signal (FIG. 4G). In contrast, motor neurons that lacked nuclear TDP-43 exhibited focal cytoplasmic accumulations of KCNQ2 (FIG. 4G). This significant difference between cases and controls was quantified by measuring the KCNQ2 signal intensity variation (FIG. 4H). These KCNQ2 accumulations exhibited substantial overlap with Ubiquitin B (UBB), suggesting that they likely reflect aggregated protein (FIG. 4I).

TABLE 1

| | Patient | | | | | Disease | | |
|---|---|---|---|---|---|---|---|---|
| Patient | Code | Mutation | Sex | Age | Ethnicity | Duration (mo) | Cause of death | PMI-cr |
| Cont 1 | 090012 | Control | M | 89 | W | n/a | Congestive heart failure | 16 |
| Cont 2 | 100028 | Control | M | 69 | W | n/a | Saphylococcal Pneumonia; Chronic | 3.25 |
| | | | | | | | Obstructive Pulmonary Disease | |
| Cont 3 | AZ170016 | Control | M | 75 | Hisp | n/a | Abdominal aortic aneurysm; | 2 |
| | | | | | | | hypertension with atherosclerosis | |
| sALS 1 | AZ200001 | sALS | M | 66 | W | 48 | ALS; respiratory failure | 18.42 |
| sALS 2 | AZ170019 | sALS | M | 69 | W | 24 | ALS | 1 |
| sALS 3 | AZ200014 | sALS | M | 82 | no data | 24 | ALS | No data |

### DISCUSSION

Understanding the functional implications of TDP-43 pathophysiology in human MNs has been a topic of intense investigation since it was first identified in ubiquitinated aggregates of postmortem brains from ALS and FTD inflicted individuals. Applicants and others have recently reported on the irregular splicing of TDP-43 target genes including STMN2, highlighting an unexpected link between TDP-43 biology and cytoskeletal architecture, and UNC13A, highlighting a link between TDP-43 and an ALS-risk locus. Both of these transcripts become mis-spliced in TDP-43 deficient cells and degraded by RNA surveillance pathways. In this study, Applicants find that nuclear reduction of TDP-43 levels yields altered KCNQ2 mRNA that escapes NMD degradation and is translated into a dysfunctional protein with severely reduced current conductance. This de novo protein variant aggregates in the ER of neurons and likely infers deficiencies associated with neuronal excitability and ER-related stress.

The mis-splicing of KCNQ2 represents a robust TDP-43 dependent event. Applicants observed it in transcriptomes of iPSC-derived spinal cholinergic MNs and cortical glutamatergic neurons in vitro, as well as in the spinal cord and motor cortex of ALS patient tissue. This event is pervasively detected in sporadic and familial ALS with known predisposition for TDP-43 aggregation, such as patients with C9orf72 mutations, while it is not detected in neurological controls or non-TDP-43 ALS cases, such as those with SOD1 mutations. As there is a paucity of reliable biomarkers for diagnosis of ALS, Applicants propose KCNQ2^{ΔE5}, which is absent in neurotypical individuals and abundant in ALS and FTD-TDP patients, as a potential biomarker of disease.

TDP-43 has hundreds of target RNAs in the nervous system. It is required for regulated alternative splicing, but also to repress the inclusion of nonconserved cryptic exons. There has been a growing appetite for the discovery of the latter in pathological conditions, in particular, because of their propensity for dramatic effects on gene expression. Notably KCNQ2 mis-splicing does not fit either of these categories: exon 5 is deeply conserved and constitutively included in KCNQ2 under normal circumstances, therefore it is neither a regulated alternative spliced locus, nor a cryptic exon. Nevertheless, like derepressed cryptic exons, our analysis suggests KCNQ2^{ΔE5} is a human-specific event. Murine CNS cells lack the UG repeat element that is likely critical for TDP-43 recruitment, and when depleted of TDP-43 do not yield this mis-splicing event. This lack of conservation is similarly observed for other TDP-43 target genes such as STMN2 and UNC13A and suggests evolutionarily divergent mechanisms for regulating these genes in neurons. It also necessitates the need for the development of humanized mouse models to evaluate the significance of these TDP-43 pathophysiological mechanisms in the context of an intact nervous model system in vivo.

Examination of neuronal nuclei with decreased TDP-43 abundance revealed that as much as 50% of KCNQ2 may be altered in affected cells, which may be an underestimation. As this analysis is based on bulk RNA-seq it may represent 100% of mis-spliced KCNQ2 in 50% of neurons, or 50% of mis-splicing in 100% of neurons. While the application of single cell technologies will illuminate the precise extent of misprocessing, the prominent aggregation of KCNQ2 protein in patient tissue suggests that this defect is pronounced at least at the terminal stages of the disease.

While TDP-43 is undoubtedly critical for the health of MNs, the question that remains unanswered is which of the multitude of transcripts, regulated by TDP-43 control important neuronal pathways that are altered in ALS patients. KCNQ2 channels mediate the repolarizing M-current, which activates to dampen neuronal excitability and limits repetitive firing following bursts of action potentials, while mutations in this gene can cause mild neonatal epilepsy to severe epileptic encephalopathy. Our finding that the TDP-43 dependent misprocessing of KCNQ2 results in non-functional protein, effectively a “dead” channel, has strong implications for neuronal excitability.

Applicants recently showed that cortical neurons with a heterozygous loss-of-function mutation in KCNQ2 associated with DEE exhibited a dyshomeostatic adaption that caused an increased bursting firing pattern. Applicants anticipate that the functional ramifications of KCNQ2^{ΔE5} in the context of TDP-43 dysfunction will be similar. Although seizures are not clinically observed in ALS patients, neuronal hyperexcitability is a broadly observed feature in both familial and sporadic disease. As TDP-43 loss-of-function effects are both gradual and heterogenous within the CNS, mis-splicing of KCNQ2 would not cause epileptic seizures, but rather disruptions in neuronal firing. Our findings provide a direct mechanistic link between TDP-43 dysfunction and decreased neuronal M-currents that may be contributing to cortical hyperexcitability seen in patients. Intriguingly, it is noteworthy that the anticonvulsant ezogabine, which is being evaluated in clinical trials as a treatment for ALS, stabilizes and increases KCNQ2-5 channels. Our data suggests that KCNQ2 may be an attractive therapeutic target for ALS. However, strategies aimed at simply restoring wildtype KCNQ2, or M-current levels will not stem the tide of KCNQ2 aggregation. Splicing modulators that can restore processing of pre-mRNA transcripts may be a fruitful alternative.

Our observation on the cytosolic accumulation of KCNQ2^{ΔE5} is an unexpected consequence of KCNQ2 misprocessing that underscores the prioritization of examining precise splice variants over genetic loss-of-function to study TDP-43 targets. It underscores a direct link between preoteotoxic ER stress and neuronal hyperexcitability acting downstream of TDP-43 dysfunction in ALS and FTD-TDP patients. Lastly, Applicants note the possibility for the de novo KCNQ2^{ΔE5} protein variant to trigger autoimmune deregulation, constituting another collateral effect of TDP-43 dysfunction that may be contributing to ALS pathophysiology.

### Materials and Methods

### Cell Lines and Cell Culture

All experiments utilizing stem cell based neuronal differentiations employed the WA01 (otherwise known as H1) human ES cell line from WiCell®. This cell line was also modified to generate KCNQ2 mutant strains to model exon 5 skipping. The stem cells were maintained on plates coated with Matrigel® Basement Membrane Matrix (Corning®, CLS354234) in mTeSR™ or mTeSR™ Plus media (StemCell Technologies, 100-0276) and passaged with Accutase (Gibco, A11105).

Chinese hamster ovary cells (CHO-K1, CRL 9618, American Type Culture Collection (ATCC), Manassas VA, USA) stably expressing human KCNQ3 (Kv7.3; GenBank accession NM_004519) were grown in F-12 nutrient mixture medium (GIBCO/Invitrogen, San Diego, CA, USA) supplemented with 10% fetal bovine serum (ATLANTA Biologicals, Norcross, GA, USA), Zeocin (100 μg/ml) and hygromycin B (600 μg/ml), penicillin (50 units·ml−1), streptomycin (50 μg·ml−1) at 37° C. in 5% CO2.

SH-SY5Y cells (CRL-2266™, ATCC, Manassas VA, USA) were grown in DMEM (Gibco, 11965092) supplemented with 15% fetal bovine serum (Takara Bio, 631106), GlutaMAX (Gibco, 35050061), penicillin (50 units·ml−1) and streptomycin (50 μg·ml−1).

Primary glial cell cultures were derived from postnatal day 0-2, CD-1 mice (Charles River). Briefly, brain cortices were dissected free of meninges in dissection buffer HBSS (Thermo Fisher), then digested with trypsin (Thermo Fisher) and DNAse I (Worthington) for 10 min at 37° C. The tissue was dissociated in glia medium: DMEM (Corning, #15-013-CV) supplemented with Glutamax, D-glucose, 10% normal horse serum (Life Technologies), and penicillin-streptomycin (Thermo Fisher). After centrifugation and resuspension, cells were filtered through a 0.45-micron cell strainer and plated on poly-D-lysine coated plates with glia media at 37° C., 5% CO2 for 2 weeks. Afterwards, glial cultures were tested for mycoplasma, dissociated for expansion, and frozen in 10% DMSO/horse serum. All animal experiments were approved and conducted in accordance with the policies and guidelines set forth by the Northwestern University Institutional Animal Care and Use Committee (IACUC35).

All cell cultures were maintained at 37° C. and 5% CO2. All cell lines were regularly tested for presence of mycoplasma using MycoAlert PLUS Detection Kit (Lonza) and determined to be mycoplasma-free.

### Generation of KCNQ2 Expression Plasmids

For experiments in FIGS. 3C-D and FIGS. 6A-B full length human KCNQ2 splice isoform 3 or 4 (NM_004518.5) was engineered in the mammalian expression vector pIRES2-EGFP or a modified vector where EGFP was substituted by CyOFP1. Site-directed mutagenesis of KCNQ2 was performed using QuikChange II XL (Agilent technologies, Santa Clara, CA, USA) to delete amino acids 231-272 to generate KCNQ2^{ΔE5}. These plasmids were transiently transfected into stably expressing KCNQ3 CHO cells by Lipofectamine transfection for functional analyses as reported previously.

For experiments in FIGS. 3E-G and FIGS. 6B-H characterizing subcellular localization of KCNQ2^{ΔE5} Applicants created a KCNQ2 fusion protein with enhanced green fluorescent protein (EGFP) or dsRed (RFP) by placing the reporters before the N-terminus of the KCNQ2 subunit (GFP-KCNQ2^{WT}, RFP-KCNQ2^{WT}, GFP-KCNQ2^{ΔE5}). An NheI-EcoRI cassette of KCNQ2 was generated, encoding for the full length human KCNQ2 splice isoform 3 or 4 (NM_004518.5) with (KCNQ2^{ΔE5}) and without (KCNQ2^{WT}) splicing mutation. A flexible linker sequence (SGRTQISSSLAQQQQQQQ) (SEQ ID NO: 34) was introduced by PCR immediately before the translation start codon (ATG) to increase protein flexibility, using the following oligonucleotides: (forward): 5′-AGCTCGCTAGCCCAGCAGCAGCAGCAGCAGCAGATGGTGCAGAAGTCGCGCAACGG CGGCGTATACC-3′ (SEQ ID NO: 35); (reverse): 5′-GCCGAATTCAGGCACCGTGCTGAGGAGGGCCG-3′ (SEQ ID NO: 36).

KCNQ2 was then subcloned into lentiviral vector (pLenti/PZ200) with a human Synapsin 1 (SYN1) promoter with NheI-EcoRI. EGFP or RFP was subcloned between the hSYN1 promotor and KCNQ2 N-terminal linker with BamHI-NheI (stop codon removed) using the following oligonucleotides:

GFP: (forward):
(SEQ ID NO: 37)
5′-AGCGGGGGATCCCCGGGTAC-3′;
(reverse):
(SEQ ID NO: 38)
5′-GCGGCTAGCGAGCTCGAGATCTGAGTCCGGCCGGACTTGTACAGCT
CGTCCATGCCGAGAGTGATCCC-3′;
RFP: (forward):
(SEQ ID NO: 39)
5′-TCGGGATCCCGTCGCCACCATGGTGGCCTCCTCCGAGGACGTCATC
AAAG-3′;
(reverse):
(SEQ ID NO: 40)
5′-GCGGCTAGCGAGCTCGAGATCTGAGTCCGGCCGGACAGGAACAGGT
GGTGGCGGCCCTCGGAG-3′.

These plasmids were used to generate lentivirus for functional and localization experiments in heterologously expressing CHO cells.

### Lentivirus Production and Transduction

TetO-Ngn2-puro (Addgene plasmid #52047) and TetO-FUW-EGFP (Addgene plasmid #30130) plasmids were gifts from Marius Wernig^{51,52}. FUW-M2rtTA (Addgene plasmid #20342) was a gift from Rudolf Jaenisch⁵³. These lentiviruses were generated by ALSTEM. Lentiviruses, including Ln-hSYN-GFP-KCNQ2^{ΔE5}, Ln-hSYN-GFP-KCNQ2^{WT} and Ln-hSYN-RFP-KCNQ2^{WT} were generated in HEK293T cells using the second-generation packaging vectors, psPAX2 and pMD2.G, as described previously^{24,54} by the Northwestern University DNA/RNA Delivery Core. CHO cells transduced with reporter fused KCNQ2 lentiviruses were patched or fixed between 3-4 days following transduction and stained with DAPI, anti-GFP, and anti-RFP primary antibodies for confocal imaging.

### Generation of KCNQ2^{ΔE5} Engineered Cell Line

Four guide RNAs (gRNAs) targeting introns 4 and 5 of KCNQ2 (Figure S4A) were designed using the CRISPR wizard with default settings on Benchling (2021, https://benchling.com). gRNA oligos were purchased from IDT with BbsI sticky ends to clone into an expression vector driven by the human U6 promotor (custom synthesis Broad Institute, Cambridge). Oligos were phosphorylated using T4 PNK (NEB #M0201S) and ligated into the BbsI-linearized pUC6-gRNA vector using T7 Ligase (NEB #M0318S). Clones containing gRNAs were verified by Sanger sequencing.

The KCNQ2^{ΔE5} engineered cell line is a derivative of WA01 human ES cells (Alias: H1, WiCell). H1 cells were transfected using the 100 μL Neon™ Transfection System (ThermoFisher Scientific, MPK10025). H1 Cells growing in mTeSR™ Plus media (StemCell Technologies, 100-0276) with 1 mM ROCK inhibitor Y-27632 were dissociated using Accutase (Gibco, A11105) treatment for 5 min at 37° C. Cells were resuspended in buffer R at a concentration of 2.5×10⁶ cells/mL. The cells were then added to a vial containing 1.4 μg of each of four gRNA vectors and 7 μg of the pSpCas9n (BB)-2A-Puro (PX462) V2.0, a gift from Feng Zhang (Addgene). The electroporated cells were immediately fed mTeSR™ Plus media containing ROCK inhibitor in a 10-cm dish. 24 hours after transfection, puromycin selection was started. Media was replaced with mTeSR™ containing 2 μg/mL of puromycin. After 24 hr of selection, the medium was aspirated and replaced. Cells were cultured for 10 days before colony picking into a 24-well plate for expansion.

Genomic DNA was extracted from puromycin-selected colonies using the DirectPCR Lysis Reagent (Viagen Biotech, 102-T) and PCR screened to confirm the presence of the intended deletion in the KCNQ2 gene. PCR products were separated and analyzed after electrophoresis on a 1% Agarose gel. Clonal cells with KCNQ2 exon 5 deletion and control cells with intact exon 5 were verified using allele-specific qPCR assays, and characterized by karyotyping at Cell Line Genetics, Inc. Oligos used for gRNA cloning include:

KCNQ2 gRNA1:
(SEQ ID NO: 41)
CTGTGCCCGGAGGCTCAGGG
KCNQ2 gRNA2:
(SEQ ID NO: 42)
GCCCAGGCCAGGAGGCTCAG
KCNQ2 gRNA3:
(SEQ ID NO: 43)
TGGTCCCAGCACAGGGACAG
KCNQ2 gRNA4:
(SEQ ID NO: 44)
CCCGTGGTCATGATGGCTTG

And genotyping primers for confirmation of exon 5 deletions are:

KCNQ2_genot_F1:
(SEQ ID NO: 45)
GTGAACGAGCCTCCCTCCCCTT
seq_KCNQ2_rvs:
(SEQ ID NO: 46)
GGCCAGATCTGTTCAGACCTG

### Neuronal Differentiations

Motor neurons (MNs): Applicants followed a previously described protocol to generate lower induced motor neurons. Briefly, WA01 cells were infected with a cocktail of three lentiviral constructs (TetO-Ngn2-Puro, TetO-GFP and rtTA, produced by ALSTEM. Media was replaced after overnight incubation in mTeSR Plus with 1 mM ROCK inhibitor Y-27632. Cells were passaged and differentiated at 70-80% confluency. On days 1~4 of the differentiation protocol, cells were grown in induction media consisting of the following minimal components: DMEM/F12 (Life Technologies, 11320-033), N2 supplement (0.5% v/v, Gibco, 17502048), 1× GlutaMAX (Gibco, 35050061), 0.1 mM MEM non-essential amino acid (Gibco, 11140050), 0.5% glucose. Media was changed daily, with the addition of the following small molecules: Day 1-10 mM SB431543 (Custom Synthesis), 200 nM LDN193189 (Custom Synthesis), doxycycline hyclate (2 mg/mL), 2 mM retinoic acid (Sigma) and 2 mM Smoothened agonist (Custom Synthesis). Day 2 to 4-puromycin (5 mg/mL), 10 mM SB431543, 200 nM LDN193189, doxycycline hyclate (2 mg/mL), 2 mM retinoic acid and 2 mM Smoothened agonist. Cells were dissociated on day 4 using Accutase (Gibco, A11105) treatment and diluted (1:2) on Matrigel coated plates. For days 4 to 7 cells were grown in media containing Neurobasal (Life Technologies 21103049) supplemented with B27 supplement (2% v/v, Gibco), 1× GlutaMAX (Gibco), 0.1 mM non-essential amino acid (Gibco), 0.5% glucose with the addition of 10 ng/ml of BDNF, CNTF and GDNF (R&D Systems). For day 7 to 10, liMNs were grown with small molecules as described above in neuronally supportive medium: Neurobasal (Life Technologies 21103049) supplemented with B27 supplement (2% v/v, Gibco), N2 supplement B (0.5% v/v, Gibco), 1× GlutaMAX (Gibco), 0.1 mM non-essential amino acid (Gibco), 0.5% glucose with the addition of 10 ng/ml of BDNF, CNTF and GDNF (R&D Systems). On day 7, cells were dissociated using Accutase and replated on glial co-cultures as described previously in medium described above. From this time onwards, half-media change was performed every 2-3 days in neuronally supportive media described above with the only addition of 10 ng/ml of BDNF, CNTF and GDNF (R&D Systems). For most experiments, neurons were co-cultured with mouse glial cells (50,000 cells/cm²) derived from postnatal brains (P0-2) as previously described, neurons were mixed with glia when replating day 7 cells 30,000 cells/cm².

Excitatory neurons: Similar to lower MNs, Applicants used previously described protocols to generate cortical-like excitatory neurons. Briefly, WA01 cells were infected with a cocktail of three lentiviral constructs (TetO-Ngn2-Puro, TetO-GFP and rtTA) produced by ALSTEM. Media was replaced after overnight incubation in mTeSR Plus with 1 mM ROCK inhibitor Y-27632. Cells were passaged and differentiated at 70-80% confluency. On days 1~4 of the differentiation protocol, cells were grown in induction media consisting of the following minimal components: DMEM/F12 (Life Technologies, 11320-033), N2 supplement (0.5% v/v, Gibco, 17502048), 1× GlutaMAX (Gibco, 35050061), 0.1 mM MEM Non-essential amino acid (Gibco, 11140050), 0.5% glucose. Media was changed daily, with the addition of the following small molecules: Day 1-10 mM SB431543 (Custom Synthesis), 200 nM LDN193189 (Custom Synthesis), doxycycline hyclate (2 mg/mL), 4 mM XAV939 (Stemgent, 0400046). Day 2 to 4-puromycin (5 mg/mL), 10 mM SB431543, 200 nM LDN193189, doxycycline hyclate (2 mg/mL), 4 mM XAV939 (Stemgent, 04-00046). Cells were dissociated on day 4 using Accutase (Gibco, A11105) treatment and diluted (1:2) on Matrigel coated plates. For days 4 to 7 cells were grown in media containing Neurobasal (Life Technologies 21103049) supplemented with B27 w/o vitA (2% v/v, Gibco), 1× GlutaMAX (Gibco), 0.1 mM non-essential amino acid (Gibco), 0.5% glucose with the addition of 10 ng/ml of BDNF, CNTF and GDNF (R&D Systems). On day 7, cells were dissociated using Accutase and replated in the appropriate formats for different experiments in medium described above. From this time onwards, half-media change was performed every 2-3 days in neuronally supportive media described above with the only addition of 10 ng/ml of BDNF, CNTF and GDNF (R&D Systems)

### RNA Extraction, Gene, and Isoform Analyses in Cells

RNA was extracted from differentiated neurons plated in 12-well plates coated with PDL/laminin using the TRIzol reagent (Invitrogen). cDNAs were synthesized with approximately 200 ng of total RNA using the SuperScript IV VILO (Invitrogen) reverse transcriptase. RT-PCR assays were performed for KCNQ2 splicing analysis and the AccuPrime Pfx DNA polymerase (Invitrogen) with 32 cycles. Gene expression was quantified by RT-qPCR using the SYBR green reagent (BioRad) using recommended PCR settings.

### Quantitative RT-PCR Analysis of Mis-Spliced KCNQ2 in Postmortem Tissue

Frontal cortex brain tissues from ALS/FTD cases were provided by the Mayo Clinic Florida Brain Bank. A descriptive characteristic table of the study cohort is included in Table 2. All cases were neuropathologically confirmed to present TDP-43 pathology, and controls confirmed to lack TDP-43 neuropathological features. Written informed consent was provided by all participants or their family members, and all protocols were approved by the Mayo Clinic Institution Review Board and Ethics Committee. Sample size was determined based on the availability of tissue in our brain bank. Levels of KCNQ2 splicing transcript variants were determined using complementary DNA (cDNA) resulting from 500 ng of RNA (RNA integrity, RIN ≥7.0) that was available from a previous study (PMID: 28637276). Quantitative real-time PCRs (qRT-PCR) were conducted using SYBR GreenER qPCR SuperMix (Invitrogen) for all samples in triplicates. qRT-PCR were run in a QuantStudio™ 7 Flex Real-Time PCR System (Applied Biosystems). Relative quantification was determined using the ΔΔCt method and normalized to the endogenous controls GAPDH and RPLP0. To compare RNA levels between controls and TDP-43 pathological confirmed cases, we used single-variable (unadjusted) and multivariable linear regression models (adjusted). Multivariable models were adjusted for age at death, sex, and RIN. KCNQ2 RNA levels analyzed on the base 2 logarithmic scale due to their skewed distributions. The regression coefficients (B) and 95% confidence intervals (CIs) were estimated and interpreted as the difference in the means, bases on the 2 logarithmic scale, between all TDP-43 pathologically confirmed cases or and the controls (reference group). P values less than 0.05 were considered statistically significant. (See Table 3 for comparisons of KCNQ2ΔE5; see also Table 4 for comparisons of KCNQ2). For associations between KCNQ2 RNA levels and pTDP-43 protein levels, pTDP-43 values were also analyzed on the base 2 logarithmic scale using single-variable and multivariable linear regression models. The multivariable model examining KCNQ2 RNA and pTDP-43 protein levels was adjusted for age, sex, and genotype. The model evaluating TDP-43 and age at disease onset was adjusted for sex, RIN and mutation status, the model assessing associations between KCNQ2 RNA and age of onset was adjusted for sex, RIN and mutation status, and the model assessing associations between KCNQ2 RNA and disease duration was adjusted for sex, age at onset, RIN, and mutation status. P values less than 0.0167 were considered statistically significant after adjusting for multiple comparisons. (See Table 5).

TABLE 2
Patient characteristics of the post-mortem cohort.

| | Controls | FTLD-TDP |
|---|---|---|
| Variable | (N = 27) | (N = 89) |
| Age at death | 81 (55, 99) | 73 (49, 99.6) |
| (years) | | |
| Sex (female) | 12 (44.4%) | 43 (48.3%) |
| Survival after | NA | 6.8 (0.6, 20.5) |
| onset (years) | | |
| Age at onset | NA | 65.8 (44.0, 85.9) |
| (years) | | |
| RIN | 9.6 (8.8, | 9.2 (6.8, 10.0) |
| | 10.0) | |

The sample median (minimum, maximum) is given for continuous variables. Information was unavailable regarding survival after onset (FTLD-TDP: N = 4), and age at onset (FTLD-TDP: N = 4). Among the FTLD-TDP cases: 47 were C9orf72 mutants, and 9 were GRN mutants.

TABLE 3
Comparisons of KCNQ2ΔE5 in frontal cortex between
FTLD-TDP cases and controls

| | | | | | Adjusting for age at | |
|---|---|---|---|---|---|---|
| | | Median | Unadjusted | | death, sex, and RIN | |
| | | (minimum, | analysis | | Regression | |
| | | maximum) | Regression | | coefficient | |
| | | levels of | coefficient | | (95% | |
| Group | N | KCNQ2ΔE5 | (95% CI) | P-value | CI) | P-value |
| Controls | 27 | 3 × 10⁻³ | 0.00 | NA | 0.00 | NA |
| | | (0, 2.52 × 10⁻⁴ | (reference) | | (reference) | |
| FTLD- | 89 | 2.35 × 10⁻³ | 4.966 | <0.0001 | 4.994 | <0.0001 |
| TDP | | (0, | (3.790, | | (3.809, | |
| | | 3.49 × 10⁻²) | 6.142) | | 6.179) | |

CI = confidence interval. Estimates, 95% CIs, and p-values result from linear regression models where the levels of KCNQ2ΔE5 in the frontal cortex were considered on the base 2 logarithm scale.
Estimates are interpreted as the difference in mean levels of KCNQ2ΔE5 in the frontal cortex (on the base 2 logarithm scale) between the given group of diseased patients and control cases. p-values ≤0.05 are considered as statistically significant after correction for multiple testing.
NA: not applicable.

TABLE 4
Comparisons of wildtype KCNQ2 in frontal cortex
between FTLD-TDP cases and controls

| | | Median | | | | |
|---|---|---|---|---|---|---|
| | | (minimum, | Unadjusted | | Adjusting for age | |
| | | maximum) | analysis | | at death, sex, and RIN | |
| | | levels of | Regression | P- | Regression | |
| | | wildtype | coefficient | value | coefficient | P- |
| Group | N | KCNQ2 | (95% CI) | | (95% CI) | value |
| Controls | 27 | 0.198 (0.054, | 0.00 | NA | 0.00 | NA |
| | | 0.417) | (reference) | | (reference) | |
| FTLD- | 89 | 0.220 (0.047, | 0.07658 | 0.6631 | −0.00124 | 0.9947 |
| TDP | | 0.806) | (−0.2707, | | (−0.3684, | |
| | | | 0.4239) | | 0.3659) | |

CI = confidence interval. Estimates, 95% CIs, and P-values result from linear regression models where the levels of wildtype KCNQ2 in the frontal cortex were considered on the base 2 logarithm scale.
Estimates are interpreted as the difference in mean levels of wildtype KCNQ2 in the frontal cortex (on the base 2 logarithm scale) between the given group of diseased patients and control cases. P-values ≤0.05 are considered as statistically significant after correction for multiple testing.
NA: not applicable.

TABLE 5
Associations of KCNQ2ΔE5 with pTDP-43 and clinical
characteristics in FTLD-TDP frontal cortex.

| Association | Unadjusted analysis | | Multivariable analyses | | Adjustment |
|---|---|---|---|---|---|
| between | Estimate | | Estimate | P- | multivariable |
| KCNQ2ΔE5 and: | (95% CI) | P-value | (95% CI) | value | analyses |
| pTDP-43 | 0.9636 | <0.0001 | 0.835 | <0.0001 | Age, sex, RIN, |
| | (0.7274, 1.200) | | (0.5990, 1.071) | | gene |
| | | | | | mutation status |
| Age at onset (yrs) | −0.0885 | 0.0005 | −0.0763 | 0.0053 | Sex, RIN, gene |
| | (−0.1368, −0.0401) | | (−0.1293, −0.02335) | | mutation |
| | | | | | status |
| Disease duration | −0.1201 | 0.0282 | −0.1028 (−0.2015, | 0.0322 | Age at onset, |
| (yrs) | (−0.227, −0.01315) | | −0.004058) | | sex, RIN, |
| | | | | | gene mutation |
| | | | | | status |

CI-confidence interval. Estimates, 95% CIs, and P-values result from linear regression models, where frontal cortex KCNQ2ΔE5 and pTDP-43 were considered on the base 2 logarithm scale. P-values ≤0.0167 are considered as statistically significant after correction for multiple testing.

### Cross-Linking and Immunoprecipitation (CLIP)/RNA Immunoprecipitation (RI) P for Nuclear RNA-Protein Isolation

SHSY5Y cells were grown in six 10 cm plates. Media was removed and cells were washed once in PBS. Plates were then placed on ice w/o their lids and irradiated once with 400 mJ/cm² using a UV 500 Crosslinker. Cells were scraped off the plate and resuspended in 2 mL PBS, 2 mL nuclear isolation buffer consisting of 1.28 M sucrose, 40 mM Tris-HCl pH 7.5, 20 mM MgCl2, 4% Triton X-100 supplemented with protease (Roche, #11836170001) and phosphatase inhibitors (Roche, #4906845001), and 2 mL water, and incubated on ice for 20 min with occasional mixing. Nuclei were pelleted by centrifugation at 2,500 g for 15 mins. The pellet was resuspended in 500 μL RIP buffer consisting of 150 mM KCl, 25 mM Tris pH 7.4, 5 mM EDTA, 0.5 mM DTT, 0.5% IGEPAL CA-630, supplemented with 100 U/mL RNasin Plus RNase Inhibitor (Promega, #N2611), as well as protease and phosphatase inhibitors. The nuclei were lysed with 15-20× strokes of a dounce homogenizer and centrifuged for 10 mins at 13,000 RPM at 4C to remove nuclear membrane and debris. Approximately 1 mg of nuclear lysate was used for each RIP assay. To prepare antibody/bead conjugates, Dynabeads protein A beads (50 μl) (Invitrogen, #10001D), were washed twice in RIP buffer, resuspended in 200 μl of RIP buffer containing 4 μg of TDP43 or IGG antibody (Proteintech, #10782-2-AP and 30000-0-AP) and incubated for 1 h at 4C under rotation. The beads were isolated magnetically and resuspended in 500 μl of IP buffer that contained 1 mg of nuclear lysate and incubated overnight at 4C under rotation. The beads were washed 4× in RIP buffer and finally in NT2 which consists of 150 mM NaCl, 50 mM Tris pH 7.4, 1 mM MgCl₂, 0.05% IGEPAL CA-630. A third of the sample was used for protein extraction performed with 50 μM glycine pH 2.8 at 23C, 500 rpm, for 20 min. The remainder of the beads were used for RNA collection. RNA was eluded twice with NT2 buffer supplemented with 0.1% SDS, 30 μg proteinase K. Each round of elution was performed, at 55C, 500 rpm, for 30 min. RNA was finally purified by phenol/chloroform extraction. RNA pellets were resuspended in water and the entire samples were used for cDNA synthesis. Applicants used semi-quantitative RT-PCR to assess the interaction between TDP-43 and KCNQ2, using GAPDH as control.

### Transcriptomic Datasets

Applicants obtained published datasets from the NCBI Gene Expression Omnibus or the European Nucleotide Archive for analyses in this study. FACS-purified MNs depleted of TDP-43 studies were reported previously: GSE121569. Purified TDP-43-high and -low neuronal nuclei datasets were reported previously: GSE126543. i³Neurons, SH-SY5Y and SK-N-DZ^{a} TDP-43 depletion studies were recently reported previously: PRJEB42763. Mouse TDP-43 depletion studies were reported previously: GSE27394. Raw sequencing data was mapped to the Human reference genome sequence (GRCh38.p13) using HISAT2. The raw reads for TDP-43 iCLIP from SH-SY5Y cells were also downloaded from E-MTAB-10297 and mapped using bowtie with parameters -a -1 10-m 1 -v 1.

Applicants also obtained 1124 RNA-seq datasets the New York Genome Center (NYGC) ALS/Target ALS Consortium. These consisted of de-identified postmortem brain samples from the motor, frontal, occipital and temporal cortices, as well as cerebellum, and cervical, thoracic, and lumbar spinal cord. Similar to other RNA-seq datasets, Applicants mapped fastq files to the Human reference genome sequence (GRCh38.p13) using HISAT2.

### Detection of KCNQ2, STMN2 and UNC13A Mis-Splicing in Postmortem RNA-Seq Datasets

A custom script was used to count reads that indicated mis-splicing. First, Applicants filtered the BAM files to only include unique, high quality mapped reads using Samtools. Applicants mined these files to identify split reads that mapped across junctions of interest with no mismatches and with at least 15 nucleotides mapped across each side of the junction. Applicants used the “Subject Group” metadata provided by the NYGC to assign sample IDs to Control, ALS or other neurological disease. Furthermore, Applicants also stratified ALS samples into Sporadic, C9orf72 expansion and Non TDP-43 using “Reported Genomic Mutations from sites”.

### Splicing Analyses

Splice junctions were mapped using the MAJIQ algorithm (2.0) under default conditions. Splice graphs and known/novel local splice variants (LSVs) were defined through use of the MAJIQ builder provided with comprehensive human gene annotations (GENCODE Release 36—GRCh38.p13) and uniquely mapped BAM files as input. The MAJIQ Quantifier was used to calculate relative abundances (Percent selected index—PSI) for all defined junctions from the perspective of sources and targets. Following this, the MAJIQ dPSI function was used to compare different conditions and the Voila methods outputted the results into tabular formats containing LSVs that underwent significant changes between specified conditions.

Custom R scripts were written to process all significant LSVs to obtain significantly changing splice junctions. Briefly, Applicants used probability_changing>0.8 and mean_dpsi_per_lsv_junction>0.1. Together, these criteria provided junctions with strong statistical likelihood of at least 10% change between conditions. MAJIQ also defined de novo junctions based on the provided GENCODE annotations. Applicants adopted a more stringent definition in which de novo junctions were also required to have a PSI<0.05 in control conditions. This allowed us to distinguish de novo splicing that was due to poor annotation of human neurons, versus those that are repressed by TDP-43. Sashimi plots were generated using the Integrative Genomics Viewer (IGV).

### Electrophysiological Recordings

CHO cell voltage clamp recordings: Whole-cell voltage clamp recordings were made from visually identified GFP-KCNQ2 expressing CHO cells using an inverted Olympus IX-73 microscope equipped with a 40× objective. Recording pipettes were made of glass capillaries using a horizontal Sutter P-1000 puller yielding a 2-3.5MΩ resistance pipette when filled with standard intracellular solution containing (in mM): 120 K-MeSO4, 10 KCl, 10 HEPES, 10 Na2-phosphocreatine, 4 Mg-ATP, 0.4 Na3-GTP, pH 7.3 adjusted with KOH; osmolality 285-290 mOsm/Kg. Bath solution contained (in mM): 135 NaCl, 3 KCl, 1.25 NaH2PO4, 1.2 MgSO4, 10 glucose, 2.4 CaCl2, 10 HEPES, pH 7.3 and was heated to 35° C. using a stage mounted heating device (ALA Scientific Instruments, Inc.). Whole-cell voltage clamp recordings were acquired using an Axopatch 200B amplifier (Molecular Devices, USA) and filtered at 5 kHz, gain 0.5 mV/pA, >1 GΩ seal on a cell. Whole-cell currents were elicited from a holding potential of −80 mV using 1000 ms depolarizing pulses (from −80 mV to +40 mV in +10 mV steps). Currents were measured in the final 100 ms of the voltage steps.

Syncropatch recordings: Automated voltage-clamp recordings were performed at room temperature using a Syncropatch 768PE (Nanion Technologies, Munich, Germany) as described previously (Vanoye et al., 2018) except that the internal solution contained 5 mM Mg-ATP. The contribution of background currents was determined by recording before and after addition of XE991 (25 mM, TOCRIS, Minneapolis, MN). Only XE991-sensitive currents were used for analysis. Whole-cell currents were elicited from a holding potential of 80 mV using 1000 ms depolarizing pulses (from 80 mV to +40 mV in +10 mV steps every 20 secs) followed by a 300 ms step to 0 mV to analyze tail currents. Cells with seal resistance 0.5 GW and series resistance 20 MW (access resistance compensation was set to 80%) were used for analysis. Peak currents were measured 999 ms after the start of the depolarizing voltage pulse and tail currents 5 ms after changing the membrane potential to 0 mV. The time-constant of activation (t) was determined by fitting currents elicited by voltage steps between 30 mV and +40 mV (50-1000 ms after start of the voltage step) to a single exponential.

### Immunohistochemistry in Postmortem Tissue

Paraffin-embedded postmortem tissue sections collected from the lumbar spinal cord of three control and three sporadic ALS patients were obtained from the VA Biorepository Brain Bank (VABBB) and analyzed by immunohistochemistry (see Table 2 for patient details). Immunohistochemistry was performed as previously described with minor alterations. Sections were first deparaffinized and rehydrated through sequential incubation (all at room temperature) in the following solutions: 3×10 min in xylene, 3×5 min in 100% EtOH, 3×3 min in 95% EtOH, 1×2 min in 75% EtOH, 1×2 min in 50% EtOH, and 5×1 min in deionized H2O. Sections were then moved into 1× Antigen Decloaker (Biocare Medical) and antigen retrieval was performed within an electronic pressure cooker (decloaking chamber) for 10 minutes at high pressure (10.2-11.6 psi) and 125° C. Samples were then cooled to room temperature for 30 minutes prior to 5 washes with deionized water. Blocking of sections was performed using 1% BSA in PBS containing 0.3% Triton X-100 for 1 hour at room temperature, prior to 5× washes with deionized water and overnight incubation with primary antibodies diluted in PBS containing 1% BSA at 4° C. Primary antibodies and dilutions used were as follows: rabbit polyclonal anti-KCNQ2 (Thermo Fisher: Cat #PA1-929, 1:100), mouse monoclonal anti-TDP-43 (Proteintech: Cat #60019-2-Ig, 1:500), mouse monoclonal anti-Ubiquitin (Santa Cruz: Cat #sc-8017, 1:100), chicken polyclonal anti-MAP2 (Abcam: Cat #ab5392, 1:5000). Following 5 additional washes with deionized water, sections were then incubated with secondary antibodies diluted in PBS containing 1% BSA for 1 hour at room temperature. Secondary antibodies and dilutions used were as follows: donkey polyclonal anti-rabbit, Alexa Fluor 488 (Invitrogen: Cat #A21206, 1:250), donkey polyclonal anti-mouse, Alexa Fluor 568 (Invitrogen: Cat #A10037, 1:250), donkey polyclonal anti-chicken, Alexa Fluor 647 (Jackson Immuno: Cat #703-606-155, 1:250). Samples were then washed 5 times with deionized water prior to a 45 second incubation with 0.3% sudan black in 70% ethanol and 15 additional rinses with deionized water. Sections were then briefly air dried and mounted using ProLong Diamond Antifade Mountant with DAPI (Invitrogen) and allowed to cure overnight at room temperature prior to imaging by confocal microscopy (see Image acquisition and analysis section for details).

### Immunocytochemistry in CHO Cells and ESC-Derived Neurons

ESC-derived neurons: ESC derived neurons were co-cultured with primary mouse glia on PDL/laminin-coated glass coverslips. Coverslips were fixed with either 2% or 3.7% formaldehyde in 4% sucrose/PBS for 15 min at room temperature and then washed three times with cold PBS prior permeabilization and blocking in 0.1% PBST with 5% normal goat serum (NGS) for 1 hour at room temperature. Primary antibodies incubated overnight at 4° C. The following primary antibodies were used: GFP (Abcam ab6673), Map2 (Millipore MAB3418, AB_94856, 1:1000), KCNQ2 (Thermo PA1-929, 1:200), Calnexin, ANK-G (Neuromab 73-146, 1:200). The following day coverslips were washed three times with cold PBS then incubated with secondary antibody for 1 hour at room temperature. The following secondary antibodies were used: Alexa 488 goat anti-chicken, Alexa 568 goat anti-mouse and Alexa 647 goat anti-rabbit (Thermo Fisher Scientific, 1:1000). Primary and secondary antibodies were diluted in PBS containing 5% normal goat serum. Cells were washed three times in PBS and then briefly in distilled water and mounted onto microscope slides.

CHO cells expressing GFP/RFP-KCNQ2 lentivirus were fixed in 3.7% formaldehyde in 4% sucrose/PBS for 10-15 minutes at room temperature and then washed three times with cold PBS prior permeabilization and blocking in 0.1% PBST with 5% normal goat serum (NGS) for 1 hour at room temperature. Primary antibodies incubated overnight at 4° C. The following primary antibodies were used: GFP (Abcam ab13970/Abcam ab6673, 1:500) and RFP (Sigma-Aldrich SAB2702202, 1:500). The following day coverslips were washed three times with cold PBS then incubated with secondary antibody for 1 hour at room temperature. The following secondary antibodies were used: Alexa 488 goat anti-chicken (check) and Alexa 555 goat anti-mouse (Thermo Fisher Scientific, 1:1000). Primary and secondary antibodies were diluted in PBS containing 5% normal goat serum. Cells were washed three times in PBS and then incubated in DAPI (Invitrogen 33342, 1:1000) in PBS for 10 minutes RT before a brief wash in distilled water. Coverslips were mounted in Fluoromount-G media (SouthernBiotech 0100-01) onto microscope slides (Superfrost Plus 12-550-15).

### Image Acquisition and Analysis

Mounted slides with post-mortem tissue were imaged using a Nikon Ti2 inverted microscope system, equipped with a Yokogawa CSU-W1 spinning disk for confocal imaging and Hamamatsu Flash 4 cameras. Mounted slides with cultured ESC-derived cortical neurons were imaged with Nikon A1 laser scanning confocal. All images were captured using a 60× oil-immersion objective (Plan Apo VC, Nikon). Cortical and spinal motor neurons were identified using MAP2 as a marker, neurons in post-mortem tissue were selected within the ventral horn of the spinal cord. Z-stacks were acquired using 0.2 μm-0.4 μm intervals, depending on the assay.

Analysis of KCNQ2 signal in heterologous expressing CHO cells and ESC-derived cortical neurons was completed from maximum intensity z-stack projections. CHO cells were selected for imaging and analysis if they had signal from both GFP-KCNQ2 and RFP-KCNQ2 transgenes. Regions of interest (ROI) were manually defined for each cell and used to compute KCNQ2 signal metrics. In ESC-derived neurons, further characterization of KCNQ2 signal was assessed in two neuronal compartments—the soma and axon initial segment (AIS). The AIS was defined as the neurite with ankyrin-G (ANKG) signal and absence of MAP2. Metrics were generated from ROIs for both compartments in cells with clearly defined AIS. For each cell, KCNQ2 signal in the soma was assigned to one of two categories: (1) diffuse KCNQ2 signal or (2) accumulated KCNQ2 signal. Neurons were then assigned into a secondary category based on KCNQ2 signal in the AIS, which was determined either KCNQ2-present (+) or KCNQ2-absent (−). Neurons from control and KCNQ2dE5 fell into three unique categories: (1) Diffuse-KCNQ2 and AIS(+), (2) Diffuse-KCNQ2 and AIS(−), or (3) Accumulated-KCNQ2 and AIS(−).

### Splice Modulating KCNQ2 ASO Data.

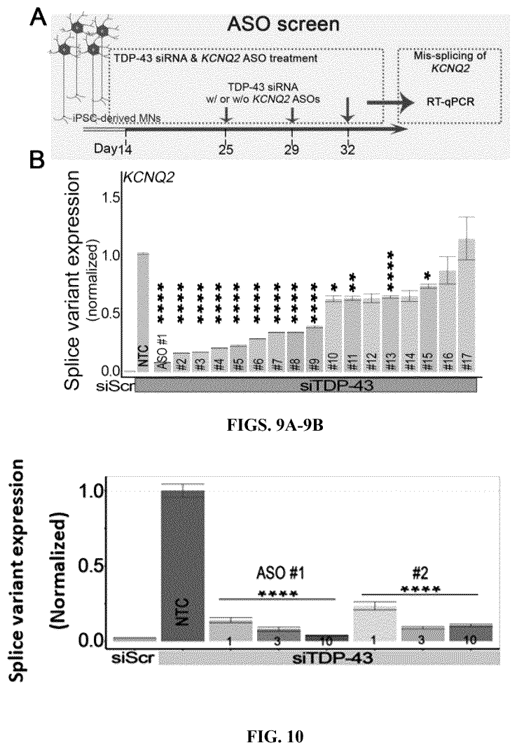

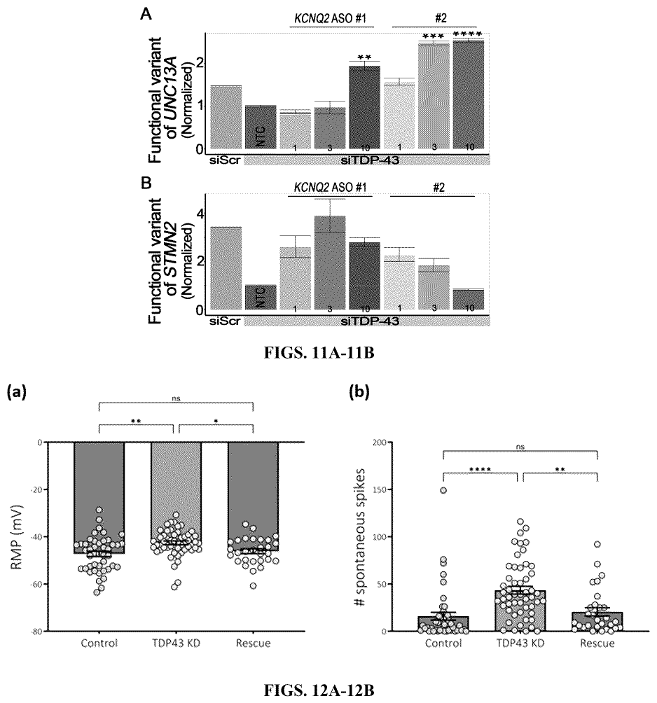

To identify effective splice-modulating ASOs for KCNQ2, Applicants designed and screened 17 ASOs that target the intron between exons 4 and 5 and the immediate downstream sequence of exon 5. The screen was performed in human induced pluripotent stem cell (iPSC)-derived spinal motor neurons (MNs) with TDP-43 or scramble siRNA treatment (FIG. 9A). Out of 17 tested ASOs, 13 ASOs significantly suppressed TDP-43 knockdown-induced mis-splicing of KCNQ2 (FIG. 9B). Applicants next selected two lead ASOs for additional dose-dependent validation experiments. The validation included three doses for each ASO, and both ASOs showed dose-dependent effects in decreasing the amount of mis-spliced KCNQ2 transcript. (FIG. 10). Previous studies demonstrate that several dozen genes are mis-spliced upon TDP-43 knockdown, including UNC13A and STMN2. Applicants therefore next explored the effect of KCNQ2 splice modulating ASOs on the expression of UNC13A and STMN2. Interestingly, both genes were upregulated upon KCNQ2 ASO treatment, although the effect for STMN2 is not significant with the current small sample size (FIGS. 11A-11B). The mechanism underlying the inter-gene regulation remains to be studied. However, the “collateral” beneficial effects of the KCNQ2 ASO treatment on other TDP-43 target genes suggest that they could be therapeutically beneficial beyond KCNQ2 itself, likely through indirect mechanisms on TDP-43 functional efficiency.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

### REFERENCES

1 Taylor, J. P., Brown, R. H., Jr. & Cleveland, D. W. Decoding ALS: from genes to mechanism. Nature 539, 197-206, doi: 10.1038/nature20413 (2016).
2 Ghasemi, M. & Brown, R. H., Jr. Genetics of Amyotrophic Lateral Sclerosis. Cold Spring Harb Perspect Med 8, doi: 10.1101/cshperspect.a024125 (2018).
3 Kim, G., Gautier, O., Tassoni-Tsuchida, E., Ma, X. R. & Gitler, A. D. ALS Genetics: Gains, Losses, and Implications for Future Therapies. Neuron 108, 822-842, doi: 10.1016/j.neuron.2020.08.022 (2020).
4 Tziortzouda, P., Van Den Bosch, L. & Hirth, F. Triad of TDP43 control in neurodegeneration: autoregulation, localization and aggregation. Nat Rev Neurosci 22, 197-208, doi: 10.1038/s41583-021-00431-1 (2021).
5 Kabashi, E. et al. TARDBP mutations in individuals with sporadic and familial amyotrophic lateral sclerosis. Nat Genet 40, 572-574, doi: 10.1038/ng. 132 (2008).
6 Sreedharan, J. et al. TDP-43 mutations in familial and sporadic amyotrophic lateral sclerosis. Science 319, 1668-1672, doi: 10.1126/science. 1154584 (2008).
7 Brown, A. L. et al. TDP-43 loss and ALS-risk SNPs drive mis-splicing and depletion of UNC13A. Nature 603, 131-137, doi: 10.1038/s41586-022-04436-3 (2022).
8 Ma, X. R. et al. TDP-43 represses cryptic exon inclusion in the FTD-ALS gene UNC13A. Nature 603, 124-130, doi: 10.1038/s41586-022-04424-7 (2022).
9 Klim, J. R. et al. ALS-implicated protein TDP-43 sustains levels of STMN2, a mediator of motor neuron growth and repair. Nat Neurosci 22, 167-179, doi: 10.1038/s41593-018-0300-4 (2019).
10 Melamed, Z. et al. Premature polyadenylation-mediated loss of stathmin-2 is a hallmark of TDP-43-dependent neurodegeneration. Nature neuroscience 22, 180-190, doi: 10.1038/s41593-018-0293-z (2019).
11 Prudencio, M. et al. Truncated stathmin-2 is a marker of TDP-43 pathology in frontotemporal dementia. The Journal of clinical investigation 130, 6080-6092, doi: 10.1172/jci139741 (2020).
12 Dittman, J. S. Unc13: a multifunctional synaptic marvel. Curr Opin Neurobiol 57, 17-25, doi: 10.1016/j.conb.2018.12.011 (2019).
13 van Es, M. A. et al. Genome-wide association study identifies 19p13.3 (UNC13A) and 9p21.2 as susceptibility loci for sporadic amyotrophic lateral sclerosis. Nature genetics 41, 1083-1087, doi: 10.1038/ng.442 (2009).
14 Menon, P. et al. Sensitivity and specificity of threshold tracking transcranial magnetic stimulation for diagnosis of amyotrophic lateral sclerosis: a prospective study. Lancet Neurol 14, 478-484, doi: 10.1016/S1474-4422 (15) 00014-9 (2015).
15 Vucic, S., Nicholson, G. A. & Kiernan, M. C. Cortical hyperexcitability may precede the onset of familial amyotrophic lateral sclerosis. Brain 131, 1540-1550, doi: 10.1093/brain/awn071 (2008).
16 Geevasinga, N. et al. Axonal ion channel dysfunction in c9orf72 familial amyotrophic lateral sclerosis. JAMA Neurol 72, 49-57, doi: 10.1001/jamaneurol.2014.2940 (2015).
17 Mogyoros, I., Kiernan, M. C., Burke, D. & Bostock, H. Strength-duration properties of sensory and motor axons in amyotrophic lateral sclerosis. Brain 121 (Pt 5), 851-859, doi: 10.1093/brain/121.5.851 (1998).
18 Kanai, K. et al. Motor axonal excitability properties are strong predictors for survival in amyotrophic lateral sclerosis. J Neurol Neurosurg Psychiatry 83, 734-738, doi: 10.1136/jnnp-2011-301782 (2012).
19 Shibuya, K. et al. Motor cortical function determines prognosis in sporadic ALS. Neurology 87, 513-520, doi: 10.1212/WNL.0000000000002912 (2016).
20 Bae, J. S., Simon, N. G., Menon, P., Vucic, S. & Kiernan, M. C. The puzzling case of hyperexcitability in amyotrophic lateral sclerosis. J Clin Neurol 9, 65-74, doi: 10.3988/jcn.2013.9.2.65 (2013).
21 Kiskinis, E. et al. Pathways Disrupted in Human ALS Motor Neurons Identified through Genetic Correction of Mutant SOD1. Cell stem cell, doi: 10.1016/j.stem.2014.03.004 (2014).
22 Wainger, B. J. et al. Intrinsic membrane hyperexcitability of amyotrophic lateral sclerosis patient-derived motor neurons. Cell Rep 7, 1-11, doi: 10.1016/j.celrep.2014.03.019 (2014).
23 Wainger, B. J. et al. Effect of Ezogabine on Cortical and Spinal Motor Neuron Excitability in Amyotrophic Lateral Sclerosis: A Randomized Clinical Trial. JAMA Neurol 78, 186-196, doi: 10.1001/jamaneurol.2020.4300 (2021).
24 Simkin, D. et al. Dyshomeostatic modulation of Ca(2+)-activated K(+) channels in a human neuronal model of KCNQ2 encephalopathy. Elife 10, doi: 10.7554/eLife.64434 (2021).
25 Vaquero-Garcia, J. et al. A new view of transcriptome complexity and regulation through the lens of local splicing variations. Elife 5, e11752, doi: 10.7554/eLife. 11752 (2016).
26 Ling, J. P., Pletnikova, O., Troncoso, J. C. & Wong, P. C. TDP-43 repression of nonconserved cryptic exons is compromised in ALS-FTD. Science 349, 650-655, doi: 10.1126/science.aab0983 (2015).
27 Brown, A. L. et al. TDP-43 loss and ALS-risk SNPs drive mis-splicing and depletion of UNC13A. Nature 603, 131-137, doi: 10.1038/s41586-022-04436-3 (2022).
28 Liu, E. Y. et al. Loss of Nuclear TDP-43 Is Associated with Decondensation of LINE Retrotransposons. Cell reports 27, 1409-1421.e1406, doi: 10.1016/j.celrep.2019.04.003 (2019).
29 Liu, E. Y. et al. Loss of Nuclear TDP-43 Is Associated with Decondensation of LINE Retrotransposons. Cell Rep 27, 1409-1421 e1406, doi: 10.1016/j.celrep.2019.04.003 (2019).
30 Brown, D. A. & Passmore, G. M. Neural KCNQ (Kv7) channels. Br J Pharmacol 156, 1185-1195, doi: 10.1111/j.1476-5381.2009.00111.x (2009).
31 Cooper, E. C. Made for “anchorin”: Kv7.2/7.3 (KCNQ2/KCNQ3) channels and the modulation of neuronal excitability in vertebrate axons. Seminars in cell & developmental biology 22, 185-192, doi: 10.1016/j.semcdb.2010.10.001 (2011).
32 Nappi, P. et al. Epileptic channelopathies caused by neuronal Kv7 (KCNQ) channel dysfunction. Pflugers Arch 472, 881-898, doi: 10.1007/s00424-020-02404-2 (2020).
33 Steinlein, O. K. & Jentsch, T. J. KCNQ2, the first gene found to be mutated in human generalized idiopathic epilepsy. Pathol Biol (Paris) 46, 683-684 (1998).
34 Biervert, C. et al. A potassium channel mutation in neonatal human epilepsy. Science (New York, N.Y 279, 403-406, doi: 10.1126/science.279.5349.403 (1998).
35 Goto, A. et al. Characteristics of KCNQ2 variants causing either benign neonatal epilepsy or developmental and epileptic encephalopathy. Epilepsia 60, 1870-1880, doi: 10.1111/epi. 16314 (2019).
36 Vanoye, C. G. et al. High-throughput evaluation of epilepsy-associated KCNQ2 variants reveals functional and pharmacological heterogeneity. JCI Insight 7, doi: 10.1172/jci.insight.156314 (2022).
37 Neumann, M. et al. Ubiquitinated TDP-43 in frontotemporal lobar degeneration and amyotrophic lateral sclerosis. Science 314, 130-133, doi: 10.1126/science.1134108 (2006).
38 Polymenidou, M. et al. Misregulated RNA processing in amyotrophic lateral sclerosis. Brain research 1462, 3-15, doi: 10.1016/j.brainres.2012.02.059 (2012).
39 Polymenidou, M. et al. Long pre-mRNA depletion and RNA missplicing contribute to neuronal vulnerability from loss of TDP-43. Nat Neurosci 14, 459-468, doi: 10.1038/nn.2779 (2011).
40 Simkin, D. et al. ‘Channeling’ therapeutic discovery for epileptic encephalopathy through iPSC technologies. Trends Pharmacol Sci 43, 392-405, doi: 10.1016/j.tips.2022.03.001 (2022).
41 Gunthorpe, M. J., Large, C. H. & Sankar, R. The mechanism of action of retigabine (ezogabine), a first-in-class K+ channel opener for the treatment of epilepsy. Epilepsia 53, 412-424, doi: 10.1111/j.1528-1167.2011.03365.x (2012).
42 Ihara, Y. et al. Retigabine, a Kv7.2/Kv7.3-Channel Opener, Attenuates Drug-Induced Seizures in Knock-In Mice Harboring Kcnq2 Mutations. PloS one 11, e0150095, doi: 10.1371/journal.pone.0150095 (2016).
43 Schirmer, L. et al. Neuronal vulnerability and multilineage diversity in multiple sclerosis. Nature 573, 75-82, doi: 10.1038/s41586-019-1404-z (2019).
44 Velmeshev, D. et al. Single-cell genomics identifies cell type-specific molecular changes in autism. Science 364, 685-689, doi: 10.1126/science.aav8130 (2019).
45 Soldovieri, M. V. et al. Decreased subunit stability as a novel mechanism for potassium current impairment by a KCNQ2 C terminus mutation causing benign familial neonatal convulsions. The Journal of biological chemistry 281, 418-428, doi: 10.1074/jbc.M510980200 (2006).
46 Vierbuchen, T. et al. Direct conversion of fibroblasts to functional neurons by defined factors. Nature 463, 1035-1041, doi: 10.1038/nature08797 (2010).
47 Zhang, Y. et al. Rapid single-step induction of functional neurons from human pluripotent stem cells. Neuron 78, 785-798, doi: 10.1016/j.neuron.2013.05.029 (2013).
48 Hockemeyer, D. et al. A drug-inducible system for direct reprogramming of human somatic cells to pluripotency. Cell Stem Cell 3, 346-353, doi: 10.1016/j.stem.2008.08.014 (2008).
49 Zufferey, R. et al. Self-inactivating lentivirus vector for safe and efficient in vivo gene delivery. J Virol 72, 9873-9880, doi: 10.1128/JVI.72.12.9873-9880.1998 (1998).
50 Limone, F. et al. Efficient generation of lower induced motor neurons by coupling Ngn2 expression with developmental cues. Cell Rep, 111896, doi: 10.1016/j.celrep.2022.111896 (2022).
51 Di Giorgio, F. P., Carrasco, M. A., Siao, M. C., Maniatis, T. & Eggan, K. Non-cell autonomous effect of glia on motor neurons in an embryonic stem cell-based ALS model. Nat Neurosci 10, 608-614, doi: 10.1038/nn1885 (2007).
52 Nehme, R. et al. Combining NGN2 Programming with Developmental Patterning Generates Human Excitatory Neurons with NMDAR-Mediated Synaptic Transmission. Cell Rep 23, 2509-2523, doi: 10.1016/j.celrep.2018.04.066 (2018).
53 Kim, D., Langmead, B. & Salzberg, S. L. HISAT: a fast spliced aligner with low memory requirements. Nat Methods 12, 357-360, doi: 10.1038/nmeth.3317 (2015).
54 Robinson, J. T. et al. Integrative genomics viewer. Nat Biotechnol 29, 24-26, doi: 10.1038/nbt. 1754 (2011).
55 Deng, H. X. et al. Mutations in UBQLN2 cause dominant X-linked juvenile and adult-onset ALS and ALS/dementia. Nature 477, 211-215, doi: 10.1038/nature10353 (2011).

1. An antisense oligonucleotide (ASO), comprising 10 to 30 nucleotides, wherein the ASO binds to a target region comprising an intronic sequence between exons 4 and 6 of a nucleic acid molecule encoding a human KCNQ2 protein.

2. The ASO of claim 1, wherein the ASO is modified or unmodified.

3. The ASO of claim 1, wherein the target region comprises a nucleic acid sequence of:
(SEQ ID NO: 19)
TGGTCATTGTGGTTTTCCCTTTCCCTGCTGATACACCCCTGTCCCTGTG
CTGGGACCAGGCTCTCACTGGCTGAGCCTGCTCCATACATCTCTTTGGG
GCCACCTGCTGGCTGCCCGTGGTCATGATGGCTTGTGGTCGAGGCGGGG
TGGTGGTTGCCAAGCTGGCGGGGGAGTGGGTGGGGAGGGCTGGGCCCCA
GAGAGCCTGAGCTCAGCCGTGGTGGGCATCTTCCTTCCTCCTGAGCTCA
GCCGTGGTGGGCATCTTCCTTCCTCCTGAGTGACCCTCCACCTGCTCCG
TGTCCCGATTATTGGGG
OR
TGGTCATTGTGGTTTTCCCTTTCCCTGCTGATACACCCCTGTCCCTGTG
CTGGGACCAGGCTCTCACTGGCTGAGCCTGCTCCATACATCTCTTTGGG
GCCACCTGCTGGCTGCCCGTGGTCATGATGGCTTGTGGTCGAGGCGGGG
TGGTGGTTGCCAAGCTGGCGGGGGAGTGGGTGGGGAGGGCTGGGCCCCA
GAGAGCCTGAGCTCAGCCGTGGTGGGCATCTTCCTTCCTCCTGAGCTCA
GCCGTGGTGGGCATCTTCCTTCCTCCTGAGTGACCCTCCACCTGCTCCG
TGTCCCGATTATTGGGG.

4. The ASO of claim 1, wherein the ASO comprise a nucleotide sequence selected from:
CACCATCACCATCACCACCA (SEQ ID NO: 21) or a pharmaceutically acceptable salt thereof; ACCATCACCATCACCACCA (SEQ ID NO: 22) or a pharmaceutically acceptable salt thereof; ATCACCATCACCACCACCA (SEQ ID NO: 23) or a pharmaceutically acceptable salt thereof; ACCATCACCACCACCACCA (SEQ ID NO: 24) or a pharmaceutically acceptable salt thereof; ATCACCATCACCATCACCA (SEQ ID NO: 25) or a pharmaceutically acceptable salt thereof; CACCACCATCATCACCAC (SEQ ID NO: 33) or a pharmaceutically acceptable salt thereof; CACCATCACCATCACCA (SEQ ID NO: 26) or a pharmaceutically acceptable salt thereof; ATCACCATCACCATCA (SEQ ID NO: 27) or a pharmaceutically acceptable salt thereof, AGGGAAAACCACAATGACCA (SEQ ID NO: 28) or a pharmaceutically acceptable salt thereof; CCATCATGACCACGGGCAGCC (SEQ ID NO: 29) or a pharmaceutically acceptable salt thereof; ATGACCACGGGCAGCCAGCA (SEQ ID NO: 30) or a pharmaceutically acceptable salt thereof; ACCACGGCTGAGCTCAGGCTCT (SEQ ID NO: 31) or a pharmaceutically acceptable salt thereof; or TCACTCAGGAGGAAGGAAGAT (SEQ ID NO: 32) or a pharmaceutically acceptable salt thereof.

5. The ASO of claim 1, comprising at least one modified internucleoside linkage.

6. The ASO of claim 5, wherein the modified internucleoside linkage is a phosphorothioate linkage.

7. The ASO of claim 6, wherein each of the internucleoside linkages in the ASO is a phosphorothioate linkage.

8. The ASO of claim 1, wherein at least one of the nucleotides in the ASO is a modified nucleotide.

9. The ASO of claim 8, wherein all of the nucleotides in the ASO are modified nucleotides.

10. The ASO of claim 8, wherein the modified nucleotide(s) is/are 2′-O-methoxyethyl (MOE) nucleotide(s).

11. The ASO of claim 1, wherein the nucleic acid molecule encoding a human KCNQ2 protein in a mRNA

12. A pharmaceutical composition comprising the ASO of claim 1 and a carrier.

13. A method of preventing KCNQ2 mis-splicing in a cell, comprising contacting the cell with the ASO of claim 1.

14. A method of treating or preventing a neurodegenerative disease in a subject, comprising administering to the subject the ASO of claim 1.

15. The method of claim 14, wherein the neurodegenerative disease involves TDP-43 cytoplasmic aggregation, TDP-43 nuclear depletion, or both.

16. The method of claim 14, wherein the neurodegenerative disease is Amyotrophic Lateral Sclerosis (ALS) or frontotemporal dementia (FTD).

17. The method of claim 13, wherein the subject is a human.


SEQUENCE LISTING (49 sequences)

SEQ ID NO: 1 - protein, 42 aa, Homo sapiens
  source 1..42: note = KCNQ2
ELVTAWYIGFLCLILASFLVYLAEKGENDHFDTYADALWWGL

SEQ ID NO: 2 - protein, 42 aa, Pan sp.; identical to SEQ ID NO: 1
  source 1..42: note = KCNQ2

SEQ ID NO: 3 - protein, 42 aa, Gorilla sp.; identical to SEQ ID NO: 1
  source 1..42: note = KCNQ2

SEQ ID NO: 4 - protein, 42 aa, unidentified; identical to SEQ ID NO: 1
  source 1..42: note = KCNQ2

SEQ ID NO: 5 - protein, 42 aa, Chlorocebus sp.; identical to SEQ ID NO: 1
  source 1..42: note = KCNQ2

SEQ ID NO: 6 - protein, 42 aa, Saimiri sp.; identical to SEQ ID NO: 1
  source 1..42: note = KCNQ2

SEQ ID NO: 7 - protein, 42 aa, Mus musculus; identical to SEQ ID NO: 1
  source 1..42: note = KCNQ2

SEQ ID NO: 8 - protein, 42 aa, unidentified; identical to SEQ ID NO: 1
  source 1..42: note = KCNQ2

SEQ ID NO: 9 - protein, 42 aa, Canis sp.; identical to SEQ ID NO: 1
  source 1..42: note = KCNQ2

SEQ ID NO: 10 - protein, 42 aa, Didelphis sp.; identical to SEQ ID NO: 1
  source 1..42: note = KCNQ2

SEQ ID NO: 11 - protein, 42 aa, unidentified
  source 1..42: note = KCNQ2
ELITAWYIGFLCLILASFLVYLAEKEDNEMFETYADALWWGL

SEQ ID NO: 12 - protein, 60 aa, synthetic construct
  source 1..60: note = WT
AVLAAGSQGNVFATSALRSLRFLQILRMIRMDRRGGTWKLLGSVVYAHSKELVTAWYIGF

SEQ ID NO: 13 - protein, 50 aa, synthetic construct
  source 1..50: note = MT
AVLAAGSQGNVFATSALRSLRFLQILRMIRMDRRGGTWKLLGSVVYAHSK

SEQ ID NO: 14 - protein, 60 aa, synthetic construct
  source 1..60: note = WT
LCLILASFLVYLAEKGENDHFDTYADALWWGLITLTTIGYGDKYPQTWNGRLLAATFTLI

SEQ ID NO: 15 - protein, 28 aa, synthetic construct
  source 1..28: note = MT
ITLTTIGYGDKYPQTWNGRLLAATFTLI

SEQ ID NO: 16 - DNA, 163 bp, synthetic construct
  source 1..163: mol_type = other DNA; note = KCNQ2
gtctatgcccacagcaaggagctggtcactgcctggtacatcggcttcctttgtctcatcctggcctcgttcctggtgtacttggcagagaagggggagaacgaccactttgacacctacgcggatgcactctggtggggcctgatcacgctgaccaccattg

SEQ ID NO: 17 - protein, 54 aa, synthetic construct
  source 1..54: note = KCNQ2
VYAHSKELVTAWYIGFLCLILASFLVYLAEKGENDHFDTYADALWWGLITLTTI

SEQ ID NO: 18 - DNA, 37 bp, synthetic construct
  source 1..37: mol_type = other DNA; note = KCNQ2
gtctatgcccacagcaagatcacgctgaccaccattg

SEQ ID NO: 19 - DNA, 311 bp, synthetic construct
  source 1..311: mol_type = other DNA; note = KCNQ2
tggtcattgtggttttccctttccctgctgatacacccctgtccctgtgctgggaccaggctctcactggctgagcctgctccatacatctctttggggccacctgctggctgcccgtggtcatgatggcttgtggtcgaggcggggtggtggttgccaagctggcgggggagtgggtggggagggctgggccccagagagcctgagctcagccgtggtgggcatcttccttcctcctgagctcagccgtggtgggcatcttccttcctcctgagtgaccctccacctgctccgtgtcccgattattgggg

SEQ ID NO: 20 - DNA, 148 bp, synthetic construct
  source 1..148: mol_type = other DNA; note = ASO that bind to the target sequences
tggtggtggtggtgatggtcatgatggtcatggtggtggtggtggtgatggtgatggtggtgatcgtggtggtggtgatggtgatggtggtggtgatgatggtggtgatggtggtgatggtgatggtggtggtgatggtgatggtgat

SEQ ID NO: 21 - DNA, 20 bp, synthetic construct
  source 1..20: mol_type = other DNA; note = ASO that bind to the target sequences
caccatcaccatcaccacca

SEQ ID NO: 22 - DNA, 19 bp, synthetic construct
  source 1..19: mol_type = other DNA; note = ASO that bind to the target sequences
accatcaccatcaccacca

SEQ ID NO: 23 - DNA, 19 bp, synthetic construct
  source 1..19: mol_type = other DNA; note = ASO that bind to the target sequences
atcaccatcaccaccacca

SEQ ID NO: 24 - DNA, 19 bp, synthetic construct
  source 1..19: mol_type = other DNA; note = ASO that bind to the target sequences
accatcaccaccaccacca

SEQ ID NO: 25 - DNA, 19 bp, synthetic construct
  source 1..19: mol_type = other DNA; note = ASO that bind to the target sequences
atcaccatcaccatcacca

SEQ ID NO: 26 - DNA, 17 bp, synthetic construct
  source 1..17: mol_type = other DNA; note = ASO that bind to the target sequences
caccatcaccatcacca

SEQ ID NO: 27 - DNA, 16 bp, synthetic construct
  source 1..16: mol_type = other DNA; note = ASO that bind to the target sequences
atcaccatcaccatca

SEQ ID NO: 28 - DNA, 20 bp, synthetic construct
  source 1..20: mol_type = other DNA; note = ASO that bind to the target sequences
agggaaaaccacaatgacca

SEQ ID NO: 29 - DNA, 21 bp, synthetic construct
  source 1..21: mol_type = other DNA; note = ASO that bind to the target sequences
ccatcatgaccacgggcagcc

SEQ ID NO: 30 - DNA, 20 bp, synthetic construct
  source 1..20: mol_type = other DNA; note = ASO that bind to the target sequences
atgaccacgggcagccagca

SEQ ID NO: 31 - DNA, 22 bp, synthetic construct
  source 1..22: mol_type = other DNA; note = ASO that bind to the target sequences
accacggctgagctcaggctct

SEQ ID NO: 32 - DNA, 21 bp, synthetic construct
  source 1..21: mol_type = other DNA; note = ASO that bind to the target sequences
tcactcaggaggaaggaagat

SEQ ID NO: 33 - DNA, 18 bp, synthetic construct
  source 1..18: mol_type = other DNA; note = ASO that bind to the target sequences
caccaccatcatcaccac

SEQ ID NO: 34 - protein, 18 aa, synthetic construct
  source 1..18: note = flexible linker sequence
SGRTQISSSLAQQQQQQQ

SEQ ID NO: 35 - DNA, 67 bp, synthetic construct
  source 1..67: mol_type = other DNA; note = oligonucleotides
agctcgctagcccagcagcagcagcagcagcagatggtgcagaagtcgcgcaacggcggcgtatacc

SEQ ID NO: 36 - DNA, 32 bp, synthetic construct
  source 1..32: mol_type = other DNA; note = oligonucleotides
gccgaattcaggcaccgtgctgaggagggccg

SEQ ID NO: 37 - DNA, 20 bp, synthetic construct
  source 1..20: mol_type = other DNA; note = Oligonucleotides GFP
agcgggggatccccgggtac

SEQ ID NO: 38 - DNA, 68 bp, synthetic construct
  source 1..68: mol_type = other DNA; note = oligonucleotides
gcggctagcgagctcgagatctgagtccggccggacttgtacagctcgtccatgccgagagtgatccc

SEQ ID NO: 39 - DNA, 50 bp, synthetic construct
  source 1..50: mol_type = other DNA; note = oligonucleotides RFP
tcgggatcccgtcgccaccatggtggcctcctccgaggacgtcatcaaag

SEQ ID NO: 40 - DNA, 63 bp, synthetic construct
  source 1..63: mol_type = other DNA; note = oligonucleotides
gcggctagcgagctcgagatctgagtccggccggacaggaacaggtggtggcggccctcggag

SEQ ID NO: 41 - DNA, 20 bp, synthetic construct
  source 1..20: mol_type = other DNA; note = KCNQ2 gRNA1
ctgtgcccggaggctcaggg

SEQ ID NO: 42 - DNA, 20 bp, synthetic construct
  source 1..20: mol_type = other DNA; note = KCNQ2 gRNA2
gcccaggccaggaggctcag

SEQ ID NO: 43 - DNA, 20 bp, synthetic construct
  source 1..20: mol_type = other DNA; note = KCNQ2 gRNA3
tggtcccagcacagggacag

SEQ ID NO: 44 - DNA, 20 bp, synthetic construct
  source 1..20: mol_type = other DNA; note = KCNQ2 gRNA4
cccgtggtcatgatggcttg

SEQ ID NO: 45 - DNA, 22 bp, synthetic construct
  source 1..22: mol_type = other DNA; note = KCNQ2_genot_F1
gtgaacgagcctccctcccctt

SEQ ID NO: 46 - DNA, 21 bp, synthetic construct
  source 1..21: mol_type = other DNA; note = KCNQ2_rvs
ggccagatctgttcagacctg

SEQ ID NO: 47 - protein, 61 aa, synthetic construct
  source 1..61: note = KCNQ2
LLGSVVYAHSKLLVTAWYIGFLCULASFLVYLAEKGENCHFDTYADALWWGLITLTTIGYG

SEQ ID NO: 48 - protein, 18 aa, synthetic construct
  source 1..18: note = KCNQ2
LLGSVVYAHITLTTIGYG

SEQ ID NO: 49 - protein, 56 aa, synthetic construct
  source 1..56: note = KCNQ2
LLGSVVYAHKLLVTAWYIFLCULASFLVYLAEKENCHFDTYADALWWLITLTTIGY